(19) United States
(12) Patent Application Publication
(10) Pub. No.: US 2006/0078532 A1
(43) Pub. Date: Apr. 13, 2006

(54) Method of prevention and treatment of Atherosclerosis, Peripheral vascular disease, Coronary artery disease, aging and age-related disorders including osteoporosis, arthritis, type 2 diabetes, dementia and Alzheimer's disease

(76) Inventor: Osemwota Sota Omoigui, Tarzana, CA (US)

Correspondence Address: Osemwota Sota Omoigui, 4019 W. Rosecrans Ave., Hawthorne, CA, 90250, US

(21) Appl. No.: 11/122,030

(22) Filed: May 5, 2005

Related U.S. Application Data
(63) Continuation-in-part of application No. 10/961,037, filed on Oct. 12, 2004.

(51) Int. Cl. A61K 31/785; A61K 39/395; A61K 36/064; A61K 31/401; A61K 31/366; A61K 31/675; A61K 31/66; A61K 31/56; A61K 31/225; A61K 31/192

(52) U.S. Cl. 424/78.14; 424/145.1; 514/423; 514/548; 514/460; 514/171; 514/571; 514/356; 514/59; 424/195.16; 514/89; 514/102

(57) ABSTRACT

This invention relates to a method for prevention and treatment of Atherosclerosis, Peripheral Vascular Disease, Coronary Artery Disease, and age-related disorders including Osteoporosis, Arthritis, Type II Diabetes, Dementia and Alzheimer's disease in a subject comprising administering to said subject a therapeutically effective dosage of each component or combination of statins, bisphosphonates, cholesterol lowering agents or techniques, interleukin-6 inhibitor/antibody, interleukin-6 receptor inhibitor/antibody, gp130 protein inhibitor/antibody, tyrosine kinases inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof, administered separately, in sequence or simultaneously. Inhibition of the signal transduction pathway for Interleukin 6 mediated inflammation is key to the prevention and treatment of atherosclerosis, peripheral vascular disease, coronary artery disease, aging and age-related disorders including osteoporosis, type 2 diabetes, dementia and some forms of arthritis and tumors. Inhibition of Interleukin 6 mediated inflammation may be achieved indirectly through regulation of endogenous cholesterol synthesis and isoprenoid depletion or by direct inhibition of the signal transduction pathway including interleukin-6 inhibitor/antibody, interleukin-6 receptor inhibitor/antibody, gp130 protein inhibitor/antibody, tyrosine kinases inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof. Said method for prevention and treatment of said disorders is based on inhibition of Interleukin-6 inflammation through regulation of cholesterol metabolism, isoprenoid depletion and inhibition of the signal transduction pathway.

CONTINUATION-IN-PART OF APPLICATION # 10/961,037 FILED ON Oct. 12, 2004

### BACKGROUND OF THE INVENTION

This invention relates to a method of prevention and treatment of atherosclerosis, peripheral vascular disease, coronary artery disease, aging and age-related disorders including osteoporosis, arthritis, type 2 diabetes, dementia and Alzheimer's disease, by inhibition of Interleukin 6 mediated inflammation. Inhibition of Interleukin 6 mediated inflammation may be achieved indirectly through regulation of endogenous cholesterol synthesis and isoprenoid depletion or by direct inhibition of the signal transduction pathway including interleukin-6 inhibitor/antibody, interleukin-6 receptor inhibitor/antibody, gp130 protein inhibitor/antibody, tyrosine kinases inhibitors/antibodies, mitogen-activated protein (MAP) kinase inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof.

Interleukin 6 mediated inflammation is the common causative origin for Atherosclerosis, Peripheral Vascular Disease, Coronary Artery Disease, aging and age-related disorders including Osteoporosis, Arthritis, Type 2 Diabetes, Dementia and Alzheimer's disease.

### Description of the Prior Art

The current theories and treatment options for Atherosclerosis, Peripheral Vascular Disease, Coronary Artery Disease, and age-related disorders including Osteoporosis, Arthritis, Type 2 Diabetes, Dementia and Alzheimer's disease are fragmented and not satisfactory. There is currently no unifying theory that links Interleukin-6 mediated inflammation as the common causative origin for all the above diseases. As such current strategies for each disease entails different medications and therapeutic procedures such as statins, aspirin, beta blockers, ACE inhibitors and angioplasty for atherosclerosis and coronary heart disease¹, statins and thrombolytics for peripheral vascular disease, oral hypoglycemics for Type 2 diabetes, bisphosphonates and calcitonin for osteoporosis, and Acetylcholinesterase inhibitors e.g. rivastigmine, donepezil and galanthamine for dementia and Alzheimer's disease.

### SUMMARY OF THE INVENTION

The present invention provides a method for the prevention and treatment of Atherosclerosis, Peripheral Vascular Disease, Coronary Artery Disease, aging and age-related disorders including Osteoporosis, Arthritis, Type 2 Diabetes, Dementia and Alzheimer's disease, in a human or other animal subject. Inhibition of the signal transduction pathway for Interleukin 6 mediated inflammation is key to the prevention and treatment of atherosclerosis, peripheral vascular disease, coronary artery disease, aging and age-related disorders including osteoporosis, type 2 diabetes, dementia and some forms of arthritis and tumors. Inhibition of Interleukin 6 mediated inflammation may be achieved indirectly through regulation of endogenous cholesterol synthesis and isoprenoid depletion or by direct inhibition of the signal transduction pathway including interleukin-6 inhibitor/antibody, interleukin-6 receptor inhibitor/antibody, gp130 protein inhibitor/antibody, tyrosine kinases inhibitors/antibodies, mitogen-activated protein (MAP) kinase inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof.

### DESCRIPTION OF THE DRAWINGS

FIG. 1. Mevalonate Synthesis

FIG. 2. Isoprenoid Synthesis

### DETAILED DESCRIPTION OF THE INVENTION

Atherosclerosis

Cardiovascular disease (CVD) is the leading cause of death and disability in developed nations and is increasing rapidly in the developing world. By the year 2020, it is estimated that CVD will surpass infectious diseases as the world's leading cause of death and disability. Atherosclerotic vascular disease (ASVD), which encompasses coronary heart disease, cerebrovascular disease, and peripheral arterial disease, is responsible for the majority of cases of CVD in both developing and developed countries². Atherosclerosis, a progressive disease characterized by the accumulation of lipids and fibrous elements in the arteries, constitutes the single most important contributor to this growing burden of cardiovascular disease. The link between lipid metabolism and atherosclerosis dominated the thinking until the 1980s³. Over the last fifteen years, however, a prominent role for inflammation in the pathogenesis of atherosclerosis has been established⁴. Now atherosclerosis is considered as an inflammation-mediated disease driven by complex interactions between leukocytes, platelets and cells of the vessel wall.

Endothelial injury is the first and crucial step in the pathogenesis of atherosclerosis. A plethora of genetically determined and epigenetic factors, such as oxidized low-density lipoprotein (LDL), free radicals (e.g., due to cigarette smoking), hypertension, diabetes mellitus, elevated plasma homocysteine, infectious microorganisms, autoimmune reactions, and combinations thereof, have been identified as etiological principles. Endothelial injury triggers inflammation with increased adhesiveness and activation of leukocytes (mainly monocytes) and platelets, which is accompanied by the production of cytokines, chemokines, vasoactive molecules and growth factors.

The hallmark of the early atherosclerotic lesion is the Cholesterol ester-laden (CE-laden) macrophage foam cell⁵. Progressive “free” cholesterol (FC) loading of lesional macrophages leads to a series of phospholipid-related adaptive responses. These adaptive responses eventually fail, leading to macrophage death. Macrophage death by either necrosis or apoptosis leads to lesional necrosis, release of cellular proteases, inflammatory cytokines, and prothrombotic molecules, which could contribute to plaque instability, plaque rupture, and acute thrombotic vascular occlusion⁶. Indeed, necrotic areas of advanced atherosclerotic lesions are known to be associated with death of macrophages, and ruptured plaques from human lesions have been shown to be enriched in apoptotic macrophages. The presence of apoptotic and necrotic macrophages in atherosclerotic lesions has been well documented in many human and animal studies^{7 8}

Currently, the inflammatory mediators implicated in the pathogenesis of atherosclerosis include cytokines, chemokines, vasoactive molecules and growth factors. The anti-inflammatory effects of statins are attributed to multifaceted mechanisms including inhibition of cell cycle progression, induction of apoptosis, reduction of cyclooxygenase-2 activity and an enhancement of angiogenesis. At the center of these mechanisms stands the ability to inhibit G protein prenylation through a reduction of farnesylation and geranylgeranylation⁹. In order to advance the current theories and thinking¹⁰, and clarify the relationship between these common illnesses, we submit our theory of the precise biochemical pathway, between cholesterol synthesis and inflammation, and between inflammation and Atherosclerosis, Peripheral Vascular Disease, Coronary Artery Disease, and age-related disorders including Osteoporosis, Arthritis, Type II Diabetes, Dementia and Alzheimer's disease. By elaborating this biochemical pathway, we will delineate the precise mechanism of the pleiotropic effects of statin and bisphosphonate drugs. The common mechanism of action and common pleiotropic effects of the Statin and Bisphosphonate drugs in addition to our identification of the unique activity of the Interleukin 6 cytokine among all the vast mediators of inflammation and the inflammatory response enabled us to reverse engineer this biochemical pathway. Each component of our theory is supported and validated by numerous research studies.

Acute Phase Response

The acute phase response occurs prior to antibody-mediated immunological defense. It occurs in response to an inflammatory response brought on by injury and trauma, neoplasm, or disordered immunological activity. A local reaction at the site of injury or infection leads to an activation of cytokines (specifically, IL-6, IL-1, TNF-Alpha, and interferons) that triggers a systemic response consisting of leukocytosis; increases in glucocorticoid production; increases in erythrocyte sedimentation rates, fever, activation of complement and clotting cascades; decreases in serum zinc and iron; and an increase in plasma levels of acute phase proteins, C-reactive protein (CRP), serum amyloid A, fibrinogen, and other proteins¹¹ Levels of cytokines involved in the acute phase response—TNF-Alpha, IL-1, IL-6, and fibrinogen—have been shown to be elevated in cases of unstable angina related to aneurysm^{12 13 14} and have been positively correlated with the risk of primary and recurrent myocardial infarction and death^{15 16 17}. The risk associated with these elevated levels remains constant even when the data is adjusted for other major risk factors: blood pressure, total and HDL cholesterol, body mass index, diabetes, alcohol use, family history, and exercise frequency¹⁵. Elevated levels of highly sensitive C-reactive protein (hs-CRP) have been related to increased risk of cardiovascular disease, myocardial infarction, and coronary artery disease (CAD) deaths among individuals with angina pectoris^{18 19 20}. Assayed levels of hs-CRP can increase 100 times over normal levels within 24-48 hours after an acute inflammatory stimulus. However, in long term prospective studies inter-individual variations in hs-CRP levels may occur over long periods of time, in the absence of trauma or acute infection²¹ Elevated levels of hs-CRP have shown a doubling of risk both for ischemic stroke in hypertensive men and women^{14 22} and for peripheral artery disease²³

Recent studies are now demonstrating that IL-6 and TNF-alpha are stronger predictors of cardiovascular disease than C-reactive protein. In the Health, Aging and Body Composition study²⁴, done at the Wake Forest University School of Medicine, the researchers tracked the medical history of the 2,225 participants for an average of 42 months after measuring their blood levels of C-reactive protein, IL-6 and TNF-alpha. People with the highest IL-6 levels were two to five times more likely to have a heart attack, stroke or other cardiovascular episode than those with the lowest levels. High blood levels of TNF-alpha increased the risk of heart disease by 79 percent and of heart failure by 121 percent. High levels of C-reactive protein increased the risk of heart failure by 160 percent compared to those with low levels, but they did not significantly raise the risk of a first stroke or heart attack.

As expected, the incidence of cardiovascular disease was high for people with the conventional risk factors—smoking, high blood pressure, high cholesterol and the like. But for participants free of those risk factors, the inflammation-related molecules were better predictors of heart disease.

Interleukin 6

Cytokines play an important role in the communication between cells of multicellular organisms. As intercellular mediators acting in nanomolar to picomolar concentrations they regulate survival, growth, differentiation and effector functions of cells. They are key players in the regulation of the immune response. Cytokines act on many different target cells (pleiotropism) and frequently affect the action of other cytokines in an additive, synergistic or antagonistic manner⁹. The Interleukin-6 family of cytokines, signaling through the common receptor subunit (glycoprotein) gp130, comprises interleukin (IL)-6, IL-11, leukemia inhibitory factor, oncostatin M, ciliary neurotrophic factor and cardiotrophin-1. Among its many functions, IL-6 plays an active role in inflammation, immunology, bone metabolism, reproduction, arthritis, neoplasia, and aging. IL-6 expression is regulated by a variety of factors, including steroidal hormones, at both the transcriptional and post-transcriptional levels. Elevated levels of IL-6 are associated with the highest risks for subclinical cardiovascular disease as well as for clinical cardiovascular disease in older men and women²⁵. Elevated levels of IL-6 are associated with a 34 percent increased likelihood of cognitive decline in older men and women²⁶. Interleukin-6 mediated inflammation contributes to bone resorption and osteoporosis by stimulating osteoclastogenesis and osteoclast activity^{27 28 29}. Elevated levels of Interleukin-6 have been observed in conditions of rapid skeletal turnover and hypercalcemia as in Paget's disease and multiple myeloma. In multiple myeloma, radiologic examinations reveals osteolytic lesion with the most common finding being diffuse osteopenia³⁰. Adhesion of multiple myeloma cells to stromal cells triggers IL-6 secretion by the stromal cells. The increased osteoclastic activity results in osteoporosis, painful osteolytic lesions and hypercalcemia characteristic of multiple myeloma³¹. In their youth, women are protected from osteoporosis because of the presence of sufficient levels of estrogen. Estrogen blocks the osteoblast's synthesis of Interleukin 6 and may also antagonize the Interleukin 6 receptors. Decline in estrogen production is often associated with osteopenia or osteoporosis in postmenopausal women^{32 33 34 35}. Inflammatory joint disease, particularly rheumatoid arthritis³⁶, is associated with bone resorption and increased synovial fluid levels of IL-6³⁷. Interleukin (IL)-6 production is considerably enhanced and associated with bone destruction in Staphylococcus aureus and mycobacterial arthritis, osteitis or osteomyelitis^{38 39 40}. During times of stress or depression, IL-6 levels are increased. In a study of older adults undergoing a chronic stressor (men and women who were caregiving for a spouse with dementia), Caregivers' average rate of increase in IL-6 was about four times as large as that of non-caregivers^{41 42}.

IL-6 transmits its biological signal through two proteins on the cell. One of them is IL-6 receptor (IL-6R), an IL-6-specific binding molecule with a molecular weight of about 80 kD. The other is a membrane-bound protein gp130 having a molecular weight of about 130 kD that is involved in non-ligand-binding signal transduction. IL-6 receptor exists not only in the membrane-bound form with transmembrane domain expressed on the cell surface but also as a soluble IL-6 receptor consisting mainly of the extracellular region. IL-6 and IL-6 receptor form the IL-6/IL-6 receptor complex, which after binding to gp130 transmits its biological signal to the cell. The important participants in the Interleukin-6 signaling pathway include the Janus kinases (JAKs) Jak1, Jak2 and Tyk2, the signal transducers and activators of transcription STAT1 and STAT3 and the tyrosine phosphatase SHP2 [SH2 (Src homology 2) domain-containing tyrosine phosphatase]. Protein kinases are a class of allosteric enzymes that possess a catalytic subunit which transfers a phosphate from ATP to one or more amino acid residues (as serine, threonine, or tyrosine) in a protein's side chain resulting in a conformational change affecting protein function, that play a role in regulating intracellular processes. JAK kinases; (abbreviation. for janus-activated kinase) is the name given to a family of non-receptor protein tyrosine kinases, comprising JAK1 (Janus kinase-1), JAK2 (Janus kinase-2), Tyk2 (non-receptor protein tyrosine kinase-2), which are widely expressed and JAK3 (Janus kinase-3) which is mainly found in cells of haematopoietic origin. STATS comprise a family of seven transcription factors that are activated by a variety of cytokines, hormones and growth factors.⁴³. Engagement of cell surface Interleukin-6 receptors activates the Janus kinase (JAK) family of tyrosine kinases, which in turn phosphorylate the cytoplasmic part of gp130, thereby creating docking sites for STAT factors STAT1 and STAT3^{44 45}. Activated STATs dimerize upon activation by JAKs and translocate to the nucleus where they bind specific DNA response elements and regulate the expression of certain genes. Following gp130 dimerization, IL-6 activates multiple signaling pathways (Ras dependent MAP Kinase cascade, STAT1-STAT3 heterodimer pathway, and STAT3 homodimer pathway)^{46 47 48} STAT3 is constitutively activated in bone marrow mononuclear cells in patients with myeloma. High levels of activated STAT3 are found in the myeloma cell line U266 known to produce and utilize IL-6 for survival⁴⁹.

A family of cytokine-inducible proteins inhibits the Jak-STAT signaling cascade providing an intracellular negative feedback regulation of cytokine-induced signal activation. These proteins have been variously termed suppressors of cytokine signaling (SOCS)⁵⁰, STAT-induced STAT inhibitors (SSI)⁵¹, cytokine-inducible SH2 containing protein (CIS), and Jak binding protein (JAB). The SOCS-protein family currently consists of CIS and SOCS-1 through 7. SOCS-protein expression is stimulated by various cytokines in a tissue specific manner. The gene expression of SOCS-1/SSI-1/JAB and SOCS-3/SSI-3/CIS-3, herein referred to as SOCS-1 and SOCS-3, are induced by IL-6 and LIF in various tissues. Both, SOCS-1 and SOCS-3 proteins bind to the JH1 domain of Jak-2 and thereby inhibit IL-6-, IL-11-, or LIF-induced tyrosine phosphorylation activity (by Jak-2) of gp130 and STAT-3⁵².

Cholesterol Metabolism

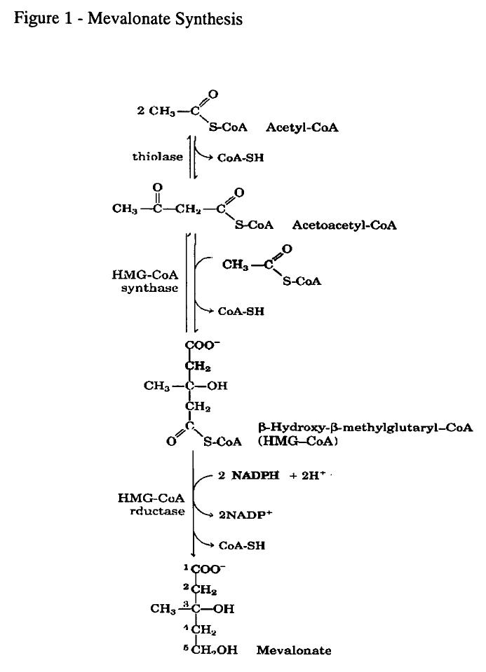

Normal healthy adults synthesize cholesterol at a rate of approximately 1 g/day and consume approximately 0.3 g/day. A relatively constant level of cholesterol in the body (150-200 mg/dL) is maintained primarily by controlling the level of de novo synthesis. The level of cholesterol synthesis is regulated in part by the dietary intake of cholesterol. Cholesterol from both diet and synthesis is utilized in the formation of membranes and in the synthesis of the steroid hormones and bile acids. The greatest proportion of cholesterol is used in bile acid synthesis⁵³. Cholesterol synthesis occurs in the cytoplasm and microsomes with initial synthesis of mevalonate from the two-carbon acetate group of acetyl-CoA. See FIG. 1 (Mevalonate Synthesis).

1. Synthesis begins when acetyl-CoA is derived from an oxidation reaction in the mitochondria and is transported to the cytoplasm

2. Two moles of acetyl-CoA are condensed, forming acetoacetyl-CoA. Acetoacetyl-CoA and a third mole of acetyl-CoA are converted to 3-hydroxy-3-methylglutaryl-CoA (HMG-CoA) by the action of HMG-CoA synthase.

3. HMG-CoA is converted to mevalonate, in a rate limiting step catalyzed by the enzyme HMG-CoA reductase, (HMGR)

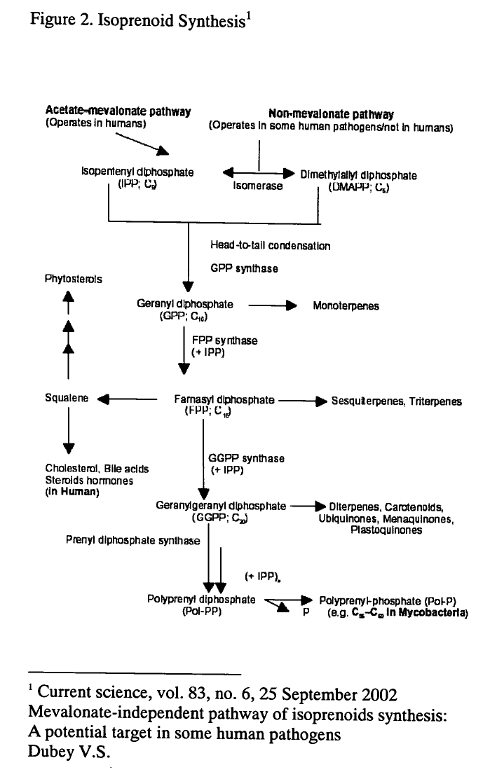

In human beings, cholesterol and isoprenoids are then synthesized via the mevalonate pathway. See FIG. 2 (Cholesterol and Isoprenoid Synthesis).

1. Mevalonate is activated by three successive phosphorylations, yielding 5-pyrophosphomevalonate

2. After phosphorylation, an ATP-dependent decarboxylation yields isopentenyl pyrophosphate, (IPP), an activated isoprenoid molecule. Isopentenyl pyrophosphate is in equilibrium with its isomer, dimethylallyl pyrophosphate, DMAPP.

3. One molecule of IPP condenses with one molecule of DMAPP to generate geranyl pyrophosphate, (GPP). This step is catalyzed by GPP synthase.

4. GPP further condenses with another IPP molecule to yield farnesyl pyrophosphate, (FPP). This step is catalyzed by FPP synthase.

5. FPP condenses with another IPP molecule to yield geranylgeranyl pyrophosphate (GGPP). This step is catalyzed by GGPP synthase

6. The head-to-tail condensation of two molecules of FPP yielding Squalene, is catalyzed by squalene synthase.

7. Squalene undergoes a two-step cyclization to yield lanosterol.

8. Lanosterol is converted to cholesterol, through a series of 19 additional reactions

There is a complex regulatory system to co-ordinate the biosynthesis of cholesterol with the availability of dietary cholesterol. The cellular supply of cholesterol is maintained at a steady level by the following mechanisms:

1. Regulation of HMGR activity and levels

2. Regulation of excess intracellular free cholesterol through the activity of acyl-CoA:cholesterol acyltransferase, (ACAT)

3. Regulation of plasma cholesterol levels via LDL receptor-mediated uptake and HDL-mediated reverse transport.

Activation of Interleukin-6 Inflammation by Isoprenoids

Cytokine receptors act through a complex signaling network involving GTPase proteins such as Ras, Rho, Rac, and Rab (particularly Rho), Janus kinases (JAKs) and the signal transducers and activators of transcription (STATs) to regulate diverse biological processes controlling immune function, growth, development and homeostasis⁵⁴.

Isoprenoids are necessary for posttranslational lipid modification (prenylation) and, hence, the function of Ras and other small guanosine triphosphatases (GTPases)⁵⁵.

GTPase proteins such as Ras, Rho, Rac, and Rab (particularly Rho) are intracellular signaling proteins that, when activated, are involved in receptor-coupled transduction of signals from extracellular stimuli to cytoplasm and the nucleus. Small GTPase proteins constitute a Ras superfamily, which is comprised of at least five major branches. Members of the Ras branch include the Ras, Rap, Ral and R-Ras family proteins^{56 57}. The Ras family regulates gene expression. The Rho branch constitutes a second major branch, with RhoA, Rac1 and Cdc42 the most studied members. The Rho family regulates cytoskeletal reorganization and gene expression. The Rab branch is the largest, and, together with members of the Arf/Sar branch, serve as regulators of intracellular vesicular transport. Ran is the sole member of its branch and is a crucial regulator of nucleo-cytoplasmic transport of proteins and RNA. The Ras superfamily proteins alternate between an inactivated GDP-bound form and activated GTP-bound form, allowing them to act as molecular switches for growth and differentiation signals. Prenylation is a process involving the binding of hydrophobic isoprenoid groups consisting of farnesyl or geranylgeranyl residues to the C-terminal region of Ras protein superfamily. Farnesyl pyrophosphate (FPP) and geranylgeranyl pyrophosphate (GPP) are metabolic products of mevalonate that are able to supply prenyl groups. The prenylation is conducted by prenyl transferases. The hydrophobic prenyl groups are necessary to anchor the Ras superfamily proteins to intracellular membranes so that they can be translocated to the plasma membrane⁵⁸. The final cell-membrane fixation is necessary for Ras proteins to participate in their specific interactions^{59 60}. The activity of the small GTPase, Rac1, plays a role in various cellular processes including cytoskeletal rearrangement, gene transcription, and malignant transformation. Small GTPases of the Ras protein superfamily stimulate the tyrosine phosphorylation and activation of the JAK family of intracellular kinases. This in turn activates the STAT family of transcription factors and results in the induction of Interleukin-6 and IL-6 receptor gene. Persistent Rac1 activity leads to the autocrine production and signal transduction of Interleukin-6³⁶. IL-6 itself may produce a delayed phosphorylation and activation of STAT3, and the JAK/STAT3 pathway is an indirect target of Ras and Rho GTPases⁶¹. Blocking the IL-6 signaling pathway inhibits Rac1-mediated STAT3-dependent gene expression. In one study⁶², constitutively active Rac1 (Rac V12) is shown to stimulate the activation of STAT3. The activity of Rac1 leads to STAT3 translocation to the nucleus coincident with STAT3-dependent gene expression⁶³. The study indicated that Rac1 induces STAT3 activation through an indirect mechanism that involves the autocrine production and action of IL-6, which is a known mediator of STAT3 response. Rac1 expression results in the induction of the IL-6 and IL-6 receptor genes and neutralizing antibodies directed against the IL-6 receptor block Rac1-induced STAT3 activation. Inhibition of nuclear factor-kappaB activation or disruption of IL-6-mediated signaling through the expression of IkappaBalpha S32AS36A and suppressor of cytokine signaling 3, respectively, blocks Rac1-induced STAT3 activation. The study also investigated whether the other Rho family members mediate STAT3 activation in an IL-6-dependent pathway. The expression of constitutively active RhoG, Cdc42, and RhoA caused the translocation from the cytoplasm to the nucleus of cotransfected STAT3-GFP. This GTPase-induced STAT3 translocation was blocked to varying degrees by neutralizing IL-6 receptor antibodies, supporting a role for autocrine IL-6 in Rho family-induced STAT3 activation These findings elucidate a mechanism dependent on the induction of an autocrine IL-6 activation loop through which Rac1 and the Rho family mediate STAT3 activation establishing a link between GTPase activity and Janus kinase/STAT signaling. Interestingly, STAT3 is persistently activated in many human cancers and transformed cell lines. In cell culture, active STAT3 is either required for transformation, enhances transformation, or blocks apoptosis.

In one study⁶⁴, leukemic cells from 50 patients with acute myeloid leukemia (AML) were analyzed for the presence of activating point mutations of the N-RAS gene using polymerase chain reaction (PCR) and differential oligonucleotide hybridization. This assay allows semiquantitative determination of the relative abundance of cells carrying N-RAS mutations. Clonal activation of N-RAS, noted in the large majority of leukemic cells of the six of these patients, was correlated significantly (p=0.0003) with the ability of these cells to express interleukin 6 (IL-6), previously shown to be expressed at high levels in approximately 30% of primary AML cells. Another study investigated the effect of a nonpeptidomimetic farnesyl transferase inhibitor R115777 in the Ras/MAPK and JAK/STAT pathways, which are implicated in survival and/or proliferation in Multiple Myeloma (MM). The phosphorylation of both STAT3 and ERK1/2 induced by IL-6 was totally blocked at 15 microM of R115777 and partially blocked when R115777 was used at 10 and 5 microM. R115777 induced (1) a significant and dose-dependent growth inhibition of the three myeloma cell lines tested; and (2) a significant and time-dependent apoptosis. R115777 also induced apoptosis in the bone marrow mononuclear cell population of four MM patients, being almost restricted to the malignant plasma cells⁶⁵.

In summary, isoprenoids farnesyl pyrophosphate (FPP) and geranylgeranyl pyrophosphate (GPP) are necessary for posttranslational lipid modification (prenylation) and, hence, the function of Ras and other small GTPase proteins such as Ras, Rho, Rac, and Rab⁵². Persistently active Rho family and Rac1 results in the activation of JAKs and subsequent tyrosine phosphorylation and activation of STAT3⁶⁶. Tyrosine phosphorylated STAT3 forms dimers that translocate to the nucleus to bind DNA target sites in responsive genes⁵⁹. IL-6 and IL-6 receptor gene induction occurs as a result of activated STAT proteins and IL-6 mediates the long-term activation of STAT3 through an autocrine loop.

Activation of Interleukin-6 Inflammation by Activated Monocytes in the Inflammatory Response to Infection and Trauma

HMG-CoA reductase generates mevalonate, the precursor of a complex series of isoprenoids molecules that are necessary for posttranslational lipid modification (prenylation) and, hence, the function of intracellular signaling proteins that, when activated, are involved in expression of Interleukin 6 mediated inflammation. Tissue injury, subsequent to a physical, chemical or biological insult, results in an inflammatory response associated with invasion of the area by immune cells, which include monocytes, T helper cells, lymphocytes, neutrophils, eosinophils, and other cells such as fibroblasts and endothelial cells. Isoprenoids are required for NADPH oxidase activity (reduced nicotinamide adenine dinucleotide phosphate) in granulocytes via low-molecular-weight (LMW) GTP-binding protein isoprenylation. Isoprenoid generation through the mevalonate pathway is a requirement for IL-8 and IL-6 induction by activated monocytic cells in vitro. One study evaluated the effects of isoprenoid depletion on the expression of proinflammatory genes in human monocyticTHP-1 cells. The researchers selected conditions under which pretreatment for 24 h with isoprenoid synthesis inhibitors (HMG-COA reductase inhibitor lovastatin or compactin at 10 microM) did not compromise cell viability but markedly suppressed hydrogen peroxide (H2O2) generation. Under these conditions interleukin-8 (IL-8) production was attenuated (by 50-90%) in response to lipopolysaccharide, granulocyte-macrophage colony-stimulating factor, and phorbol myristate acetate. Coincubation of reductase inhibitor-treated cells with mevalonate prevented the attenuation of IL-8 production by reductase inhibitors. The effects of isoprenoid depletion on cytokine production were selective: IL-1 beta generation was not inhibited but the production of IL-6 and IL-8 was concomitantly suppressed. IL-8 induction was suppressed at least in part through attenuation of the increase in mRNA in stimulated cells. The study authors concluded that isoprenylation inhibitors have the potential to alter monocyte proinflammatory function⁶⁷. In another study, fluvastatin decreased (and mevalonate rescued) signaling molecules within membrane rafts in monocytes in parallel with effects on tyrosine phosphorylation events. In addition, Fcgamma receptor mediated immune complex trafficking, activation of MAP kinases (ERK and p38), and downstream inflammatory mediator release (MMP-1 and IL-6) were blocked by fluvastatin. The study authors concluded that HMG-CoA reductase inhibition alters immune receptor signaling in monocytes by disrupting membrane rafts essential for the initiation of signal transduction⁶⁸. Another study explored the role of mevalonate inhibitors in the activation of nuclear factor kappa B (NF kappa B) and the induction of inducible nitric oxide synthase (iNOS) and cytokines (TNF-alpha, IL-1beta, and IL-6) in rat primary astrocytes, microglia, and macrophages. Lovastatin and sodium phenylacetate (NaPA) were found to inhibit Lipopolysaccharide (LPS) and cytokine-mediated production of NO and expression of iNOS in rat primary astrocytes; this inhibition was not due to depletion of end products of mevalonate pathway (e.g., cholesterol and ubiquinone). The authors stated that reversal of the inhibitory effect of lovastatin on Lipopolysaccharide (LPS)-induced INOS expression by mevalonate and farnesyl pyrophosphate and reversal of the inhibitory effect of NaPA on LPS-induced iNOS expression by farnesyl pyrophosphate suggests a role of farnesylation in the LPS-mediated induction of iNOS. The inhibition of LPS-mediated induction of iNOS by FPT inhibitor II, an inhibitor of Ras farnesyl protein transferase, suggests that farnesylation of p21(ras) or other proteins regulates the induction of iNOS. Inhibition of LPS-mediated activation of NF kappa B by lovastatin, NaPA, and FPT inhibitor II in astrocytes indicates that the observed inhibition of iNOS expression is mediated via inhibition of NF kappa B activation. In addition to iNOS, lovastatin and NaPA also inhibited LPS-induced expression of TNF-alpha, IL-1beta, and IL-6 in rat primary astrocytes, microglia, and macrophages. The authors concluded that their study delineates a novel role of the mevalonate pathway in controlling the expression of iNOS and different cytokines in rat astrocytes, microglia, and macrophages that may be important in developing therapeutics against cytokine- and NO-mediated neurodegenerative diseases⁶⁹.

Bacterial infection as typified by periodontal disease is associated with inflammation and the inflammatory response, with generation of isoprenoids by activated monocytes. Bacteria also directly synthesize isoprenoid molecules by a mevalonate—independent (non-MVA) pathway (see FIG. 1). The synthesis of IPP and DMAPP via the non-MVA pathway starts with the formation of 1-deoxy-Dxylulose-5-phosphate (DOXP) by two glycolytic intermediates, pyruvate and glyceraldehyde-3-phosphate⁷⁰. These isoprenoids may be involved in the cell-wall biosynthesis and may also play a role in direct activation of biologically active mediators⁷¹. Periodontal disease is characterized by adherence and colonization of the tooth enamel and root surface by saccharolytic, aerobic Streptococcus species. and other bacteria. This sets the stage for Fusobacterium nucleatum to coaggregate with these early colonizers and to permit late colonizers, including dental pathogens, to eventually form a biofilm. These complex interactions result in the release of factors that lead to tooth decay⁷². In a landmark study in Finland, Matilla et al examined the role of chronic bacterial infections as risk factors for coronary heart disease. The association between poor dental health and acute myocardial infarction was investigated in two separate case-control studies of a total of 100 patients with acute myocardial infarction and 102 controls selected from the community at random. Dental health was graded by using two indexes, one of which was assessed blind. Based on these indexes dental health was significantly worse in patients with acute myocardial infarction than in controls. The association remained valid after adjustment for age, social class, smoking, serum lipid concentrations, and the presence of diabetes⁷³. More recently, these results were confirmed in studies in the United States, Canada, Great Britain, Sweden, and Germany. In another study, Morrison et al found that people with periodontal disease had a factor of 2 higher risk of dying from cardiovascular disease. By comparison smokers only had a 60% increased risk⁷⁴. Meyer et al showed that c-reactive proteins and pro-inflammatory cytokines are released during periodontal flare-ups and capable of eliciting effects associated with atherosclerosis and coronary heart disease⁷⁵. The presence of oral infections is also associated with cerebrovascular disease, stroke⁷⁶, preterm births⁷⁷, osteoporosis⁷⁸ and type 2 diabetes. One study evaluated 113 Pima Indians with both diabetes and periodontal disease. The study found that when their periodontal infections were treated, the management of their diabetes markedly improved⁷⁹.

Inhibition of Cholesterol Pathway by Stains

The main effect of statins is the decrease of serum level of low-density lipoprotein (ILDL) cholesterol, due to the inhibition of intracellular cholesterol biosynthesis. A minor effect is the decrease of serum triglycerides. Statins inhibit HMG-CoA reductase and decrease the production of mevalonate, geranyl pyrophosphate, and farnesyl pyrophosphate, and subsequent products on the way to construction of the cholesterol molecule. Thus, statins could inhibit inflammation, by inhibition of the cholesterol pathway and intracellularly interfering with Ras superfamily protein function⁸⁰. Ikeda et al.⁸¹ recently showed that statins decrease matrix metalloproteinase-1 expression through inhibition of Rho. Statin therapy has been demonstrated to provide significant reductions in non-high-density lipoprotein cholesterol, and to decrease cardiovascular morbidity and mortality.

Inhibition of Cholesterol Pathway by Bisphosphonates

Recent findings suggest that alendronate and other N-containing bisphosphonates inhibit the isoprenoid biosynthesis pathway and interfere with protein prenylation, as a result of reduced geranylgeranyl diphosphate levels. One study⁸² utilizing High-performance liquid chromatography (HPLC) analysis of products from a liver cytosolic extract, identified farnesyl disphosphate (FPP) synthase as the mevalonate pathway enzyme inhibited by bisphosphonates. Recombinant human farnesyl diphosphate synthase was inhibited by alendronate with an IC(50) of 460 nM (following 15 min preincubation). Alendronate did not inhibit isopentenyl diphosphate isomerase or GGPP synthase. Recombinant farnesyl diphosphate synthase was also inhibited by pamidronate (IC(50)=500 nM) and risedronate (IC(50)=3.9 nM), negligibly by etidronate (IC50=80 microM), and not at all by the non-nitrogen-containing bisphosphonate clodronate. In another study, a wide range of bisphosphonates, were found to have a significant correlation between potency for inhibition of recombinant human FPP synthase in vitro and anti-resorptive potency in vivo, suggesting that this enzyme is the major pharmacologic target of these drugs. The most potent anti-resorptive bisphosphonates such as zoledronic acid and risedronate are very potent inhibitors of FPP synthase, with IC50 values as low as 3 nM and 10 nM respectively. Inhibition of FPP synthase prevents the formation of FPP and its derivative GGPP. These isoprenoid lipids are necessary for the post-translational lipid modification (prenylation) of small GTPase proteins such as Ras, Rho, Rac, and Rab. The effects of nitrogen-containing bisphosphonates on osteoclasts can be overcome by addition of components of the mevalonate pathway, which bypass the inhibition of FPP synthase and restore protein prenylation. In particular, geranylgeraniol (a cell-permeable form of GGPP) prevents inhibition of resorption by nitrogen-containing bisphosphonates in vitro.⁸³. One study aimed to evaluate cholesterol and lipoprotein serum levels in patients with Paget's bone disease treated with intravenous pamidronate. The study included 20 consecutive patients (mean age, 67.6±11.0 years) with Paget's bone disease for at least 1 year, who needed intravenous amino bisphosphonate treatment; 12 patients with inactive Paget's bone disease served as controls. The patients with active Paget's bone disease underwent three cycles (every 3 months) of treatment with 60 mg of intravenous pamidronate. Controls were given a saline infusion following the same administration schedule. In all subjects total alkaline phosphatase (total ALP), bone alkaline phosphatase (bone ALP), total cholesterol (TC), tryglycerides (TG), and high- and low-density lipoprotein cholesterol (HDL-C and IDLC, respectively) were measured before infusions (pamidronate or saline) at baseline and at 3-month intervals up to 9 months. In the control group no significant changes were observed through the study period for any of the biochemical parameters. In the pamidronate-treated patients, both bone ALP and total ALP significantly fell at the end of the study. In patients with active treatment, at the end of the study period HDL-C significantly (P<0.05) increased by 10.3%, whereas LDLC significantly (P<0.05) decreased by 5.5%. In these patients TC showed a negative trend without reaching statistical significance, whereas the HDL-C/LDL-C ratio rose 16.2% above the basal value and TC/HDL-C decreased by 12.5%. The researchers concluded that, pamidronate given intravenously seems to be able to induce a prolonged shifting in circulating cholesterol from the IDL-C to the HDLC that is associated with a weak decrease in total cholesterol, thus producing a possible improvement in the atherosclerotic risk index⁸⁴.

Atherosclerosis and Interleukin 6

Macrophage uptake of oxidized low-density lipoprotein (Ox-LDL) is a hallmark of the early atherosclerotic lesion, and may be mediated by Interleukin-6. Incubation of IL-6 with MPM or IL-6 administration in mice increased macrophage Ox-LDL degradation and CD36 mRNA expression. Angiotensin II (Ang II) plays an important role in atherogenesis. Ang II increases macrophage cholesterol accumulation and foam cell formation, increases contraction of blood vessels and induces hypertrophy and hyperplasia of vascular smooth muscle cells (VSMC). Ang II significantly increases the expression of IL-6 mRNA and protein in vascular smooth muscle, in a dose-dependent manner. The induction of IL-6 expression by Ang II is dependent on intracellular Ca²⁺, tyrosine phosphorylation, and mitogen-activated protein kinase (MAPK)⁸⁵. Ang II administration to apolipoprotein E-deficient atherosclerotic mice increases Ox-LDL degradation, CD36 mRNA expression, and CD36 protein expression by their peritoneal macrophages (MPMs). Ang II treatment of IL-6-deficient mice did not affect their MPM Ox-LDL uptake and CD36 protein levels. Furthermore, injection of IL-6 receptor antibodies in mice during Ang II treatment reduced macrophage Ox-LDL uptake and CD36 expression⁸⁶. Enzymatic, nonoxidative modification transforms low density lipoprotein (LDL) to an atherogenic molecule (E-LDL) that activates complement and macrophages and is present in early atherosclerotic lesions. E-LDL accumulates in human vascular smooth muscle cells (VSMC), where it stimulates the expression of gp130, the signal-transducing chain of the IL-6 receptor (IL-6R) family, and the secretion of Interleukin-6⁸⁷. IL-6/sIL-6R provokes marked up-regulation of gp130 mRNA and surface protein expression in VSMC. This is accompanied by secretion of IL-6 by the cells, so that an autocrine stimulation loop is created. In the wake of this self-sustaining system, there is a selective induction and secretion of monocyte chemotactic protein-1 (MCP-1), up-regulation of ICAM-1, and marked vascular smooth muscle proliferation⁸⁸. Interleukin-6 (IL-6) induces proliferation of vascular smooth muscle cells and the release of monocyte chemoattractant protein-1 (MCP-1). In one study, treatment with IL-6 caused rapid increase in the c-myc mRNA level of cultured vascular smooth muscle cells. IL-6 also stimulated DNA synthesis and proliferation of the cells significantly and dose-dependently at concentrations of more than 10 U/ml. The authors concluded that IL-6 may be important in the proliferation of VSMC, which is a key event in the development of atherosclerosis⁸⁹. Another study investigated IL-6 mRNA expression in atherosclerotic arteries from patients undergoing surgical vascularization, utilizing reverse transcription polymerase chain reaction (RT-PCR) and in situ hybridization analyses. In RT-PCR analysis, the atherosclerotic arteries showed 10- to 40-fold levels of IL-6 mRNA expression over the non-atherosclerotic artery. In in-situ hybridization analysis, IL-6 gene transcripts were observed in the thickened intimal layer of atherosclerotic lesions. These results strongly suggest the involvement of IL-6 in the development of human atherosclerosis⁹⁰. Thrombin is a potent mitogen for vascular smooth muscle cells (VSMCs) and plays an important role in the progression of atherosclerosis. Thrombin induces IL-6 mRNA and protein expression in a dose-dependent manner. Pharmacological inhibition of extracellular signal-regulated protein kinase (ERK), p38 mitogen-activated protein kinase (MAPK), or epidermal growth factor receptor (EGF-R) suppresses thrombin-induced IL-6 expression⁹¹. IL-6 increases the number of platelets in the circulation⁹² and activates platelets through arachidonic acid metabolism in vitro⁹³ IL-6 is reported to increase plasma fibrinogen and decrease free protein S concentration. These IL-6-induced modifications of platelet and the coagulant phase of the clotting mechanism may lead to pathological thrombosis and instability of plaque⁹⁴. IL-6 stimulation of vascular smooth muscle cells occurs via the JAK/STAT signaling pathway. In one study, Rat VSMC were stimulated with IL-6 in the presence or absence of a JAK 2 inhibitor, and the activation of STAT 3 (by Western), MCP-1 (by ELISA) and DNA synthesis (by (3)H-thymidine incorporation) was determined. IL-6 rapidly induced phosphorylation of STAT 3 in a dose- and time-dependent manner with a peak expression at 30 min. IL-6 also stimulated MCP-1 protein production and DNA synthesis dose dependently. 50 microM of AG490, a specific JAK 2 inhibitor, partially inhibited STAT 3 activation and MCP-1 production, with near complete inhibition of DNA synthesis. The authors concluded that the JAK/STAT pathway partially mediates IL-6-induced MCP-1 production and DNA synthesis in rat VSMC. The researchers further stated that these studies implicate a role of the JAK/STAT pathway in the development of vascular disease and atherosclerosis⁹⁵. Levels of IL-6 are significantly higher in patients with dyslipidemia IIa and IIb biochemically confirmed, and IL-6 levels are significantly correlated to intima-media complex thickness⁹⁶.

Statins and Interleukin 6

The ability of HMG-CoA reductase inhibitors to lower C-reactive protein levels has recently brought into question the mechanisms of action of the statin drugs. Because these medications lower incidences of acute cardiovascular events as well as decreasing morbidity and mortality well before the effects of lowered LDL cholesterol can be expected to occur, questions have been asked about whether they may work independently of LDL-lowering mechanisms. One study examined the effects of atorvastatin on soluble adhesion molecules, interleukin-6 (IL-6) and brachial artery endothelial-dependent flow mediated dilatation (FMD) in patients with familial (FH) and non-familial hypercholesterolemia (NFR)⁹⁷. A total of 74 patients (27 FH and 47 NFH) were recruited. Fasting lipid profiles, soluble intercellular adhesion molecule-1 (sICAM-1), soluble vascular-cellular adhesion molecule-1 (sVCAM-1), E-selectin, IL-6 and FMD were measured at baseline, 2 weeks, 3 and 9 months post-atorvastatin treatment (FH—80 mg/day, NFH—10 mg/day). In both groups, compared to baseline, sICAM-1 levels were significantly reduced at 2 weeks, further reduced at 3 months and maintained at 9 months (P<0.0001). The IL-6 levels were significantly reduced at 3 months and 9 months compared to baseline for FH (P<0.005) and NFH (P<0.0001). In both groups, the FMD at 2 weeks was higher than baseline (P<0.005), with progressive improvement up to 9 months. FMD was negatively correlated with sICAM-1 and IL-6. The authors concluded that both low and high doses of atorvastatin lead to early progressive improvement in endothelial function in patients with primary hypercholesterolemia and that sICAM-1 and IL-6 levels reflect endothelial dysfunction in these patients.

Bisphosphonates and Interleukin 6

Because of various modes of action observed in studies, bisphosphonates have been classified into two groups. Bisphosphonates (such as clodronate and etidronate) that closely resemble pyrophosphate—a normal byproduct of human metabolism—are incorporated into adenosine triphosphate (ATP) analogues, which create compounds that are believed to build up and lead to osteoclast death⁹⁸. The newest generation of bisphosphonates, which contain nitrogen (such as pamidronate, alendronate, risedronate, and ibandronate), are believed to inhibit protein prenylation (post-translational modification) within the mevalonate pathway. The mevalonate pathway is responsible for the biosynthesis of cholesterol, other sterols, and isoprenoid lipids. Isoprenoid lipids are key in the prenylation of intracellular signaling proteins (GTPases) that, when activated, regulate a number of processes, including osteoclast activity. It is believed that by impeding the function of these regulatory proteins, bisphosphonates block osteoclast functioning and cause apoptosis⁹⁹

In patients with Paget's disease of bone, bisphosphonate therapy is associated with a significant reduction of Interleukin-6 soluble receptor (sIL-6R) serum levels¹⁰⁰. Bisphosphonates inhibit the production of pro-inflammatory cytokine interleukin-6 in tumoral cell lines of human osteoblastic phenotype (MG63 and SaOs cells), and in peripheral blood mononuclear cells (PBMC)¹⁰¹. Bisphosphonates also inhibit IL-1 and TNF-alpha stimulated IL-6 release in cultures of human osteoblastic osteosarcoma cells¹⁰². Osteoblasts exposed to small amounts of bisphosphonate elaborate a soluble inhibitor, which interferes with osteoclast formation and development¹⁰³. Bisphosphonates prevent apoptosis of murine osteocytic MLO-Y4 cells, whether it is induced by etoposide, TNF-alpha, or glucocorticoid dexamethasone¹⁰⁴. Pamidronate and other bisphosphonates inhibit the production by osteoblasts of the inflammatory cytokine interleukin-6, a growth factor essential to myeloma cells¹⁰⁵.

Atherosclerosis and Statins

Changes in intima-media thickness (IMT) and arterial lumen diameter-as measured by B-mode high-resolution ultrasonography and quantitative coronary angiography, respectively—are currently the only surrogate markers for progression of atherosclerotic disease recognized by regulatory authorities in the United States and Europe. Because atherosclerosis is a disease of the arterial wall, the ability of B-mode ultrasonography to provide visualization of IMT offers significant advantages over angiography. These advantages, as well as the safety and noninvasiveness of B-mode ultrasonography, have led to increasing use of this imaging technique in observational studies and interventional studies of lipid-lowering agents over the last decade. These observational studies clearly demonstrated an association between carotid IMT and atherosclerotic disease. Of the interventional studies, the recent Arterial Biology for the Investigation of the Treatment Effects of Reducing Cholesterol (ARB[I° F-R) trial found that use of atorvastatin 80 mg daily for aggressive lowering of plasma low-density lipoprotein cholesterol (LDL-C) concentrations to below current target levels was associated with significant IMT regression compared with results obtained with less aggressive plasma LDL-C lowering.¹⁰⁶¹⁰⁷

Atheroscloerosis and Bisphosphonates

Measurement of carotid arterial intima-media thickness (IMT) using B-mode ultrasonography is a noninvasive and powerful tool to evaluate early atherosclerotic lesions^{108 109 110 111 112}. In one study the effect of etidronate treatment on carotid arterial intima-media thickness was prospectively examined in 57 subjects with type 2 diabetes associated with osteopenia. After 1 yr of therapy with cyclical etidronate (200 mg/day for 2 weeks every 3 months), intima-media thickness showed a decrease (mean±SE, −0.038±0.011 mm), which was significantly different from a change in 57 control subjects (0.023±0.015 mm; P<0.005). Cardiovascular parameters were not changed after etidronate treatment. The authors concluded that etidronate in clinical dosage may have an antiatherogenic action, at least in type 2 diabetes, although its mechanisms remain to be elucidated¹¹³. In another study, administration of ethane-1-hydroxy-1,1-diphosphonate (EHDP) to swine with pre-established atherosclerosis resulted in lower lesion calcium concentration, smaller lesions and a decrease in the area of lesions involved in necrosis. Atherosclerosis was developed in Yorkshire swine by balloon catheter-injury to the abdominal aorta, followed by a high cholesterol-high lipid (HL) diet for 4 months. The administration of EHDP (20 mg/kg/day) was begun after these 4 months and continued for 5 additional months along with the atherogenic diet. Other swine were ballooned and fed HL diet for nine months. Morphometric analysis showed that the extent of lesions, expressed as ratio of intima to media was significantly less (P less than 0.05) in the EHDP-treated HL swine, compared to the HL diet-only group. The ratio of lesion areas showing lipid-rich necrotic debris to the area of media was also significantly smaller (P less than 0.05). Biochemical analysis showed that the lesion from the HL drug-treated group contained significantly less (P less than 0.05) calcium compared to that from the HL diet only. Finally, there was significant correlation between average lesion area and average lesion calcium concentration (P less than 0.02) for both groups. While the effect of EHDP on lesion size and calcium concentration has been previously reported for various species such as rabbit and monkey, the authors concluded that this study is believed to be the first where a beneficial effect of EHDP on one of the most serious complications of atherogenesis—necrosis—has been documented¹¹⁴.

Type 2 Diabetes and Interleukin 6

Circulating levels of interleukin-6 (IL-6) are raised in insulin resistant states such as obesity, impaired glucose tolerance (IGT), and type 2 diabetes mellitus (DM). Growing evidence suggests that IL-6 is not only produced by fat cells but is also capable of inducing insulin resistance in these cells. The expected result of this in vivo, would be to increase adipose mass and subsequently body mass index (BMI). The IL-6 -174G>C common functional gene variant has consistently been associated with increased plasma IL-6, insulin resistance, and increased cardiovascular risk¹¹⁵. In another study, the authors determined whether elevated levels of the inflammatory markers interleukin 6 (IL-6) and C-reactive protein (CRP) are associated with development of type 2 DM in healthy middle-aged women. The Women's Health Study, is an ongoing US primary prevention, randomized clinical trial initiated in 1992. From a nationwide cohort of 27 628 women free of diagnosed DM, cardiovascular disease, and cancer at baseline, 188 women who developed diagnosed DM over a 4-year follow-up period were defined as cases and matched by age and fasting status with 362 disease-free controls. Incidence of confirmed clinically diagnosed type 2 DM by baseline levels of IL-6 and CRP. Study results showed that baseline levels of IL-6 (P<0.001) and CRP (P<0.001) were significantly higher among cases than among controls. The relative risks of future DM for women in the highest vs lowest quartile of these inflammatory markers were 7.5 for IL-6 (95% confidence interval [CI], 3.7-15.4) and 15.7 for CRP (95% CI, 6.5-37.9). Positive associations persisted after adjustment for body mass index, family history of diabetes, smoking, exercise, use of alcohol, and hormone replacement therapy; multivariate relative risks for the highest vs lowest quartiles were 2.3 for IL-6 (95% CI, 0.9-5.6; P for trend=0.07) and 4.2 for CRP (95% CI, 1.5-12.0; P for trend=0.001). Similar results were observed in analyses limited to women with a baseline hemoglobin A(1c) of 6.0% or less and after adjustment for fasting insulin level. The authors concluded that elevated levels of CRP and IL-6 predict the development of type 2 DM, and the data support a possible role for inflammation in diabetogenesis.

Type 2 Diabetes and Bisphosphonates

Advanced glycation end products (AGE), senescent macroprotein derivatives form at an accelerated rate in diabetes and induce angiogenesis through overgeneration of autocrine vascular endothelial growth factor (VEGF). In this study, effects of incadronate disodium, a nitrogen-containing bisphosphonate on AGE-elicited angiogenesis in vitro, were studied. Incadronate disodium was found to completely inhibit AGE-induced increase in DNA synthesis as well as tube formation of human microvascular endothelial cells (EC). Furthermore, incadronate disodium significantly prevented transcriptional activation of nuclear factor-kappab and activator protein-1 and the subsequent up-regulation of VEGF mRNA levels in AGE-exposed EC. FaFresyl pyrophosphate, but not geranylgeranyl pyrophosphate, was found to completely restore the anti-angiogenic effects of incadronate disodium on EC. These results suggest that incadronate disodium could block the AGE-signaling pathway in microvascular EC through inhibition of protein farnesylation. The authors concluded that Incadronate disodium may be a promising remedy for treatment of patients with proliferative diabetic retinopathy¹¹⁶. Charcot neuroarthropathy has been recognized for over 130 years and yet it remains a major cause of morbidity for patients with diabetes mellitus and a continuing challenge for physicians. The underlying cause is thought to be trauma in a neuropathic foot that leads to a complex series of pathological processes culminating in bone and joint destruction and subsequent deformity. A study was undertaken to study the effect of pamidronate, a bisphosphonate, in the management of acute diabetic Charcot neuroarthropathy. Altogether 39 diabetic patients with active Charcot neuroarthropathy from four centers in England were randomized in a double-blind placebo-controlled trial. Patients received a single infusion of 90 mg of pamidronate or placebo (saline). Foot temperatures, symptoms and markers of bone turnover (bone specific alkaline phosphatase and deoxypyridinoline crosslinks) were measured over the 12 months, in 10 visits. All patients also had standard treatment of the Charcot foot. Mean age of the study group (59% Type 2 (non-insulin-dependent) diabetes mellitus) was 56.3±10.2 years. The mean temperature difference between active and control groups was 3.6±1.7 degrees C. and 3.3±1.4 degrees C., respectively. There was a fall in temperature of the affected foot in both groups after 2 weeks with a further reduction in temperature in the active group at 4 weeks (active and placebo vs baseline; p=0.001; p=0.01, respectively), but no difference was seen between groups. An improvement in symptoms was seen in the active group compared with the placebo group (p<0.001). Reduction in bone turnover (means±SEM) was greater in the active than in the control group. Urinary deoxypyridinoline in the pamidronate treated group fell to 4.4±0.4 nmol/mmol creatinine at 4 weeks compared with 7.1±1.0 in the placebo group (p=0.01) and bone-specific alkaline phosphatase fell to 14.1±1.2 u/l compared with 18.6±1.6 u/l after 4 weeks, respectively (p=0.03). The authors concluded that the bisphosphonate, pamidronate, given as a single dose leads to a reduction in bone turnover, symptoms and disease activity in diabetic patients with active Charcot neuroarthropathy¹¹⁷.

Type II Diabetes and Statins

Type 2 diabetes is associated with a substantially increased risk of cardiovascular disease, but the role of lipid-lowering therapy with statins for the primary prevention of cardiovascular disease in diabetes is inadequately defined. One study aimed to assess the effectiveness of atorvastatin 10 mg daily for primary prevention of major cardiovascular events in patients with type 2 diabetes without high concentrations of LDL-cholesterol. 2838 patients aged 40-75 years in 132 centers in the UK and Ireland were randomized to placebo (n=1410) or atorvastatin 10 mg daily (n=1428). Study entrants had no documented previous history of cardiovascular disease, an LDL-cholesterol concentration of 4.14 mmol/L or lower, a fasting triglyceride amount of 6.78 mmol/L or less, and at least one of the following: retinopathy, albuminuria, current smoking, or hypertension. The primary endpoint was time to first occurrence of the following: acute coronary heart disease events, coronary re-vascularisation, or stroke. Analysis was by intention to treat. The trial was terminated 2 years earlier than expected because the pre-specified early stopping rule for efficacy had been met. Median duration of follow-up was 3.9 years (IQR 3.0-4.7). 127 patients allocated placebo (2.46 per 100 person-years at risk) and 83 allocated atorvastatin (1.54 per 100 person-years at risk) had at least one major cardiovascular event (rate reduction 37% [95% CI −52 to −17], p=0.001). Treatment would be expected to prevent at least 37 major vascular events per 1000 such people treated for 4 years. Assessed separately, acute coronary heart disease events were reduced by 36% (−55 to −9), coronary re-vascularisations by 31% (−59 to 16), and rate of stroke by 48% (−69 to −11). Atorvastatin reduced the death rate by 27% (−48 to 1, p=0.059). No excess of adverse events was noted in the atorvastatin group. The study authors determined that Atorvastatin 10 mg daily is safe and efficacious in reducing the risk of first cardiovascular disease events, including stroke, in patients with type 2 diabetes without high LDL-cholesterol. The researchers stated that no justification is available for having a particular threshold level of LDL-cholesterol as the sole arbiter of which patients with type 2 diabetes should receive statins. The authors concluded that debate about whether all people with this disorder warrant statin treatment should now focus on whether any patients are at sufficiently low risk for this treatment to be withheld¹¹⁸.

Osteoporosis and Interleukin 6

Osteoporosis is a condition that is common with aging and especially in post-menopausal women. The etiology has often been ascribed to abnormalities in calcium metabolism. However many patients with osteopenia/osteoporosis have in common pain and inflammation and many inflammatory pain syndromes have osteopenia/osteoporosis as an accompanying feature¹¹⁹. A notable example is the osteoporosis that is often present in Complex Regional Pain Syndrome/Reflex sympathetic dystrophy (CRPS-I/RSD)¹²⁰. Interleukin-6 mediated inflammation has been shown to contribute to the process of bone remodeling. This it does by stimulating osteoclastogenesis and osteoclast activity¹²¹. Elevated levels of Interleukin-6 have been observed in conditions of rapid skeletal turnover and hypercalcemia as in Paget's disease and multiple myeloma¹²². In multiple myeloma, radiologic examinations reveals osteolytic lesion and the most common finding is diffuse osteopenia¹²³. Adhesion of multiple myeloma cells to stromal cells triggers IL-6 secretion by the stromal cells¹²⁴. This results in increased osteoclastic activity that in turn results in osteoporosis, painful osteolytic lesions and hypercalcemia characteristic of multiple myeloma¹²⁵. In their youth, women are protected from osteoporosis because of the presence of sufficient levels of estrogen. Estrogen blocks the osteoblast's synthesis of Interleukin 6. Estrogen may also antagonize the interleukin 6 receptors. Decline in estrogen production is often associated with osteopenia/osteoporosis in postmenopausal women. Estrogen's ability to repress IL-6 expression was first recognized in human endometrial stromal cells¹²⁶. Additional clues came from the observations that menopause or ovariectomy resulted in increased IL-6 serum levels¹²⁷, increased IL-6 mRNA levels in bone cells¹²⁸, and increased IL-6 secretion by mononuclear cells^{129 130 131}. Further evidence for estrogen's ability to repress IL-6 expression is derived from studies, which demonstrated that estradiol inhibits bone marrow stromal cell and osteoblastic cell IL-6 protein and mRNA production in vitro¹³² and that estradiol was as effective as neutralizing antibody to IL-6 in suppressing osteoclast development in murine bone cell cultures¹³³ or in ovariectomized mice¹³⁴.

Osteoporosis and Bisphosphonates

Bisphosphonates are inorganic chemical compounds that bind to hydroxyapatite in bone and prevent osteoclastic absorption of bone. Nitrogen-containing bisphosphonates (N-BPs) are potent inhibitors of bone resorption widely used in the treatment of osteoporosis and other bone degrading disorders including Paget's disease of bone, hypercalcemia associated with malignancy, metastatic bone diseases, such as breast cancer, multiple myeloma, and arthritis¹³⁵ . At the tissue level, N-BPs reduce bone turnover and increase bone mass and mineralization. This is measured clinically as an increase in bone mineral density and bone strength and a decrease in fracture risk. N-BPs localize preferentially at sites of bone resorption, where mineral is exposed, are taken up by ostoclasts and inhibit osteoclastic activity. At the molecular level, N-BPs inhibit an enzyme in the cholesterol synthesis pathway, farnesyl diphosphate synthase. As a result, there is a reduction in the lipid geranylgeranyl diphosphate, which prenylates GTPases required for cytoskeletal organization and vesicular traffic in the osteoclast, leading to osteoclast inactivation^{137 138}.

Osteoporosis and Statins

3-hydroxy-3-methylglutaryl coenzyme A reductase inhibitors (statins) have been shown to stimulate bone formation in laboratory studies, both in vitro and in vivo. Statin use in most, but not all observational studies is associated with a reduced risk of fracture, particularly hip fracture, even after adjustment for the confounding effects of age, weight and other medication use. This beneficial effect has not been observed in clinical trials designed to assess cardiovascular endpoints¹³⁹. Men using statin drugs are more likely to have a greater BMD of the spine (p<0.005), and men who receive statin drugs for more than 2 yr are approximately half as likely to develop osteoporosis. A similar effect is observed in women taking statins for any length of time¹⁴⁰. Statin use in women is associated with a 3% greater adjusted BMD at the femoral neck, and BMD tends to be greater at the spine and whole body¹⁴¹. Nitrogen-containing bisphosphonate drugs inhibit the mevalonate pathway, preventing the production of isoprenoids, which consequently results in the inhibition of osteoclast formation and osteoclast function. Statins decrease the hepatic biosynthesis of cholesterol by blocking the mevalonate pathway, and can affect bone metabolism in vivo through effects on osteoclastic bone resorption. The ability of statin compounds to inhibit bone resorption is directly related to HMG-CoA reductase activity¹⁴².

Arthritis and Interleukin-6

Interleukin-1 (IL-1), a cytokine produced by chondrocytes and other cells in the joint, plays an important role in cartilage degradation by stimulating the synthesis of degradative enzymes that inhibit the production of proteoglycans. Other cytokines that appear to act synergistically with IL-1 to promote matrix breakdown are tumor necrosis factor-alpha (TNF-alpha) and interleukin-6 (IL-6). During times of stress or inflammation IL-6 levels are increased. Inflammatory joint disease, particularly rheumatoid arthritis¹⁴³, is associated with increased synovial fluid levels of IL-6¹⁴⁴. Although Osteoarthritis has previously been considered a non-inflammatory form of arthritis, there are changes that occur within the joint that are associated with inflammation. Inflammation is aggravated by the introduction of bone and cartilage breakdown products into the synovial fluid. Cells in the synovium phagocytize these products, resulting in chronic, low-grade inflammation. Consequently, the synovial membrane becomes thickened. Inflammation of the synovial membrane may be absent in the earlier stages of Osteoarthritis; however, as the disease progresses, some degree of synovitis usually exists. Once mild synovial inflammation is established, the synovium becomes a source of cartilage-degrading enzymes (e.g., MMPs) and cytokines, including IL-1, IL-6, and TNF-alpha. These substances diffuse through the synovial fluid and cause further degradation of articular cartilage. IL-1 and TNF-alpha stimulate the chondrocytes to produce more degrading enzymes, and the process continues in a vicious cycle. IL-1, IL-6, and TNF-alpha are believed to be the main cytokines linked to the disease process.

Arthritis and Bisphosphonates

Pamidronate has resulted in pain reduction in patients with osteoarthritis (with and without osteoporosis) in our clinic, via its anti-inflammatory properties resulting in a subsequent reduction of bone resorption and inflammatory bone pain¹⁴⁵. The quick onset of pain relief observed in our patients can only be attributed to its anti-interleukin-6 effect. Several literature abound documenting the anti-interleukin-6 effect of bisphosphonates. Bisphosphonates inhibit the production of pro-inflammatory cytokine interleukin-6 in tumoral cell lines of human osteoblastic phenotype (MG63 and SaOs cells), and in peripheral blood mononuclear cells (PBMC)¹⁴⁶. Pamidronate infusion has significantly decreased the mean serum levels of Interleukin-6 in patients with advanced solid tumors and bone metastases¹⁴⁷. Pamidronate and other bisphosphonates inhibit the production by osteoblasts of the inflammatory cytokine interleukin-6, a growth factor essential to myeloma cells¹⁴⁸. In patients with Paget's disease of bone, bisphosphonate therapy is associated with a significant reduction of Interleukin-6 soluble receptor (sIL-6R) serum levels¹⁴⁹. Bisphosphonates also inhibit IL-1 and TNF-alpha stimulated IL-6 release in cultures of human osteoblastic osteosarcoma cells¹⁵⁰. Osteoblasts exposed to small amounts of bisphosphonate elaborate a soluble inhibitor, which interferes with osteoclast formation and development¹⁵¹.

Furthermore, bisphosphonates prevent apoptosis of murine osteocytic MLO-Y4 cells, whether it is induced by etoposide, TNF-alpha, or the glucocorticoid dexamethasone¹⁵². In a recent study appearing in the journal, Clinical & Experimental Rheumatology Masuda-Aiba et al observed that a new third-generation bisphosphonate, YM529, represents a candidate treatment for arthritis¹⁵³. The authors report that prophylactic or therapeutic treatment of animals with experimental arthritis with YM529 suppressed the severity of disease and suggest that YM529 may act on arthritic joints locally to prevent inflammation. These data are consistent with previous clinical studies investigating the efficacy of other bisphosphonates in patients with rheumatoid arthritis. The authors concluded that although further experiments are necessary to elucidate the underlying mechanisms, YM529 deserves consideration as a treatment for this disease.

Arthritis and Statins

MMP-9 or Gelatinase B, a member of the matrix metalloproteinase family (MMPs), plays important roles in physiological events such as tissue remodeling and in pathological processes that lead to destructive bone diseases, including osteoarthritis and periodontitis. In addition to its effect on the increase of total bone mass, statin (an HMG-CoA reductase inhibitor) suppresses the expression of MMPs. In this study, the researchers proposed that simvastatin reduces MMP-9 expression in osteoblasts and HT1080 fibrosarcoma cell line. Gelatin zymography, Western blot analysis and reverse transcriptase-PCR were used to investigate the effects of simvastatin on MMP-9 in primary calvaria cells, U2-OS osteosarcoma cells, and HT1080 fibrosarcoma cells. The results from gelatin zymography and Western blot analysis revealed that simvastatin suppressed MMP-9 activity in these cells in concentration- and time-dependent manners. The effective concentrations of simvastatin were 100-500 nM, 5-15 microM, and 2.5-10 microM in primary calvaria, U2-OS, and HT1080 cells, respectively. The authors concluded that collectively, these results suggest that simvastatin is a potent drug for inhibition of MMP-9 expression in osteoblastic cells and HT1080 fibrosarcoma cells¹⁵⁴. In another study, the researchers postulated that 3-Hydroxy-3-methylglutaryl-CoA reductase inhibitors (statins) exert favorable effects on lipoprotein metabolism, but may also possess anti-inflammatory properties. The authors explored the activities of simvastatin, a lipophilic statin, in a Th1-driven model of murine inflammatory arthritis. They reported in this study that simvastatin markedly inhibited not only developing but also clinically evident collagen-induced arthritis in doses that were unable to significantly alter cholesterol concentrations in vivo. Ex vivo analysis demonstrated significant suppression of collagen-specific Th1 humoral and cellular immune responses. Moreover, simvastatin reduced anti-CD3/anti-CD28 proliferation and IFN-gamma release from mononuclear cells derived from peripheral blood and synovial fluid. Proinflammatory cytokine production in vitro by T cell contact-activated macrophages was suppressed by simvastatin, suggesting that such observations have direct clinical relevance. The authors concluded that these data clearly illustrate the therapeutic potential of statin-sensitive pathways in inflammatory arthritis¹⁵⁵. In one study, the authors set out to clarify whether the inhibition of sterol or nonsterol derivatives arising from mevalonate biotransformation plays a major role in the in vivo anti-inflammatory action of statins¹⁵⁶. Hepatic synthesis of all these derivatives was inhibited in mice by administered statins, whereas squalestatin inhibited only sterol derivatives. Using a short-term treatment schedule, the authors found that statins reduced the hepatic activity of 3-hydroxy-3-methylglutaryl coenzyme A reductase without affecting blood cholesterol. This treatment inhibited lipopolysaccharide- and carrageenan-induced pouch leukocyte recruitment and the exudate production of interleukin-6, monocyte chemotactic protein-1, and RANTES. Coadministration of mevalonate reversed the effect of statin on leukocyte recruitment. The inhibition of sterol synthesis by squalestatin did not have any anti-inflammatory effect, indicating that the biosynthesis of nonsterol compounds arising from mevalonate is crucial for the in vivo regulation of cytokine and chemokine production by statins. The authors concluded that inhibition by statins may account for the reported anti-inflammatory effects of these drugs and may provide a biochemical basis for the recently reported effects of statins in the prevention of cardiovascular disease and mortality.

Dementia, Alzheimer's Disease and Interleukin 6

Vascular (formerly Arteriosclerotic) Dementia (MID, Multi-infarct dementia) is characterized by a history of transient ischemic attacks with brief impairment of consciousness, fleeting pareses, or visual loss. The dementia may also follow a succession of acute cerebrovascular accidents or, less commonly, a single major stroke. Some impairment of memory and thinking then becomes apparent. Onset, which is usually in later life, can be abrupt, following one particular ischemic episode, or there may be more gradual emergence. The dementia is usually the result of infarction of the brain due to vascular diseases, including hypertensive cerebrovascular disease. The infarcts are usually small but cumulative in their effect. Vascular dementia can occur with other types of dementia such as Alzheimer's disease. Compared with Alzheimer's disease, vascular dementia can affect distinct parts of the brain and particular abilities may remain relatively unaffected. Alzheimer's disease affects the entire brain. Symptoms of vascular dementia remain steady for a while and then suddenly decline. In Alzheimer's disease the decline is more constant.

Alzheimer's disease (AD), the most common form of dementia, is a progressive, degenerative disorder of the central nervous system. Interleukin 6 mediated inflammation play a role in several age-related diseases, including Alzheimer's disease. The Health, Aging and Body Composition Study¹⁵⁷ enrolled 3,031 black and white men and women, with an average age of 74. The researchers took blood levels of interleukin-6 (IL-6), C-reactive protein and tumor necrosis factor and then repeated the tests two years later. A battery of mental tests was also given to evaluate concentration, memory, language and other measures of cognitive functioning, both at the start and two years later. After adjusting for age and other factors, they found that those who had the highest levels of inflammation—whose blood levels of IL-6 and C-reactive protein were in the highest one-third—had more cognitive decline compared to those whose blood levels of those substances were in the lower third. If their IL-6 result was high, they were 34 percent more likely to have cognitive decline than those whose scores on the tests were in the lower third. If their C-reactive protein levels were in the top third, they were 41 percent more likely to have cognitive decline than those in the lower third. Although those who suffered cognitive decline also had higher levels of tumor necrosis factor, the differences weren't statistically significant. The study found no relationship between the use of non-steroidal anti-inflammatory drugs (NSAIDs) and inflammation levels. This is not surprising as NSAIDs inhibit cyclooxygenase and affect prostaglandin synthesis but have no effect on IL-6 inflammation.

Dementia, Alzheimer's Disease and Statins

Increased circulating cholesterol has been long linked to an increased risk of coronary artery disease (CAD), and is now linked to an increased risk of developing Alzheimer s disease (AD). The neuropathologic link between CAD and AD manifests as increased incidence of cerebral senile plaques in both disorders. In one study, the researchers showed that AD-like neuropathology occurred in the brains of cholesterol-fed rabbits; including increased beta-amyloid (A beta)¹⁵⁸. The major hallmarks of AD include selective neuronal cell death and the presence of amyloid deposits and neurofibrillary tangles. Apolipoprotein E (ApoE) has also been shown to co-localize with these neuropathological lesions. Putative pathological functions or “risk-factor activities” of ApoE-epsilon4 include its role in promoting amyloid accumulation, neurotoxicity, oxidative stress and neuro fibrillary tangles. ApoE has been shown essential for amyloid beta-peptide fibrillogenesis and deposition, a defining pathological feature of this disease. The human ApoE gene has three alleles (epsilon2, epsilon3, epsilon4)-all products of the same gene. The epsilon3-allele accounts for the majority of the ApoE gene pool (approximately 70-80%), the epsilon4-allele accounts for 10-15% and the epsilon2 allele for 5-10%. Inheritance of the epsilon4-allele strongly increases the risk for developing AD at an earlier age. ApoE mRNA is most abundant in the liver followed by the brain, where it is synthesized and secreted primarily by astrocytes. ApoE protein and mRNA are further detected in cortical and hippocampal neurons in humans. ApoE gene expression is induced by brain injury in some neurons and upregulated in astrocytes during aging. In AD, an increased ApoE mRNA was reported in the hippocampus. The risk for AD has been reported to correlate with transcriptional activity of the ApoE gene. Binding sites for putative transcriptional factors (TF), such as AP-1, AP-2 and NF-kappaB, are present in the ApoE promoter. The promoter also contains sites for the inflammatory response transcription factors IL-6 RE-BP, MED1, STAT1 and STAT2¹⁵⁹.

Because astrocytes and microglia represent the major source of extracellular apoE in brain, one study investigated apoE secretion by glia. The authors determined that protein prenylation is required for apoE release from a continuous microglial cell line, primary mixed glia, and from organotypic hippocampal cultures. Using selective protein prenylation inhibitors, apoE secretion was found to require protein geranylgeranylation. This prenylation involved a protein critical to apoE secretion, not apoE proper. ApoE secretion could also be suppressed by inhibiting synthesis of mevalonate, the precursor to both types of protein prenylation, using hydroxyl-3-methylglutaryl coenzyme A reductase inhibitors (statins). The authors stated that recent reports have described the beneficial effects of statins on the risk of dementia. The authors further stated that their finding that protein geranylgeranylation is required for apoE secretion in the brain parenchyma provides another contributing mechanism to explain the effective properties of statins against the development of dementia. They concluded that in this model, statin-mediated inhibition of mevalonate synthesis, an essential reaction in forming geranylgeranyl lipid, would lower extracellular levels of parenchymal apoE. Because apoE has been found necessary for plaque development in transgenic models of Alzheimer's disease, suppressing apoE secretion by statins could reduce plaques and, in turn, improve cognitive function¹⁶⁰.

Statins have been reported to mediate changes in neuronal survival and cytoskeleton, including the microtubule-associated protein tau, a major constituent of the tangles. In one study to determine the effect of statin on the cytoskeleton, the authors challenged rat primary neuron cultures by lovastatin and determined the metabolite that is critical for structural integrity and survival of neurons. During the blockade of 3-hydroxy-3-methylglutaryl-coenzyme A reductase, the neuritic plaque was affected and eventually was completely destroyed. This process was not part of the execution phase of apoptosis and was marked by alterations in the microfilament and microtubule system. The distribution and phosphorylation of protein tau changed. Immunoblot analysis and indirect immunofluorescence revealed a transient increase in tau phosphorylation, which ceased during the execution of apoptosis. The researchers determined that all of these effects could be linked to the lack of the geranylgeranylpyrophosphate intermediate. Inhibition of the geranylgeranylation of Rho family GTPases (geranylgeranyl-transferase I) evoked similar changes in neurons. The researchers stated that these data and their findings that statin treatment reduced the membrane-bound fraction of RhoA-GTTase in neurons suggest that reduced levels of functional small G proteins are responsible for the observed effects. They concluded that their data demonstrates that lovastatin concentrations that are able to suppress not only cholesterol but also geranylgeranylpyrophosphate formation may evoke phosphorylation of tau reminiscent of preclinical early stages of Alzheimer's disease and, when prolonged, apoptosis¹⁶¹.

An observational study of 1037 postmenopausal women with coronary heart disease enrolled in the Heart and Estrogen/progestin Replacement Study (participants at 10 of 20 centers), was undertaken to determine whether serum lipoprotein levels, the 4-year change in serum lipoprotein levels, and the use of statin drugs are associated with cognition in older women without dementia. The Modified Mini-Mental State Examination was administered at the end of the study after 4 years of follow-up. Women whose score was less than 84 points (>1.5 SDs below the mean) were classified as having cognitive impairment. Lipoprotein levels (total, high-density lipoprotein, and low-density lipoprotein [LDL] cholesterol and triglycerides) were measured at baseline and at the end of the study; statin use was documented at each visit. Compared with women in the lower quartiles, women in the highest LDL cholesterol quartile at cognitive testing had worse mean plus minus SD Modified Mini-Mental State Examination scores (93.7 plus minus 6.0 vs 91.9 plus minus 7.6; P=0.002) and an increased likelihood of cognitive impairment (adjusted odds ratio, 1.76; 95% confidence interval, 1.04-2.97). A reduction in the LDL cholesterol level during the 4 years tended to be associated with lower odds of impairment (adjusted odds ratio, 0.61; 95% confidence interval, 0.36-1.03) compared with women whose levels increased. Higher total and LDL cholesterol levels, corrected for lipoprotein(a) levels, were also associated with a worse Modified Mini-Mental State Examination score and a higher likelihood of impairment, whereas high-density lipoprotein cholesterol and triglyceride levels were not associated with cognition. Compared with nonusers, statin users had higher mean plus minus SD Modified Mini-Mental State Examination scores (92.7 plus minus 7.1 vs 93.7 plus minus 6.1; P=0.02) and a trend for a lower likelihood of cognitive impairment (odds ratio, 0.67; 95% confidence interval, 0.42-1.05), findings that seemed to be independent of lipid levels. The authors concluded that high LDL and total cholesterol levels are associated with cognitive impairment, and lowering these lipoprotein levels may be a strategy for preventing impairment¹⁶². Another study examined the association between the use of lipid-lowering agents (LLAs) and dementia, adjusting for other markers of health, and investigated factors associated with LLA use. The authors performed a cohort study of LLA use and a case-control study of dementia in relation to LLA use, in a secondary analysis of the Canadian Study of Health and Aging( a nationally representative population-based survey of Canadians 65 years and older). To examine features associated with statin use, the authors evaluated data on 2305 people for whom health information, drug use, and cognitive status were known. To examine the relationship between LLA use and dementia, the authors selected incident cases of dementia (n=492, of whom 326 had Alzheimer disease) that occurred between the first and second waves of the study. Control subjects were 823 persons examined during the first and second phases of the Canadian Study of Health and Aging who had no cognitive impairment. Results from the study showed that use of LLAs was significantly (P<0.001) more common in younger (65-79 years) than in older (>or =80 years) people. It was not associated with other factors indicating a healthy lifestyle, but was associated with a history of smoking and hypertension. Use of statins and other LLAs reduced the risk of Alzheimer disease in subjects younger than 80 years, an effect that persisted after adjustment for sex, educational level, and self-rated health (odds ratio, 0.26; 95% confidence interval, 0.08-0.88). There was no significant effect in subjects 80 years and older. The researchers concluded that while the possibility of indication bias in the original observations cannot be excluded, it was not demonstrated in LLA use in this study. Lipid-lowering agent use was associated with a lower risk of dementia, and specifically of Alzheimer disease, in those younger than 80 years¹⁶³.

Dementia, Alzheimer's Disease and Bisphosphonates

There is very little literature on the use of bisphosphonates in patients with dementia or Alzheimer's disease. In a clinical case report of primary hyperparathyroidism in an 89-year-old woman causing profound neuropsychiatric symptoms, the use of bisphosphonate therapy led to marked but temporary improvements in her mental state¹⁶⁴. Considering the role of Cholesterol in atherosclerosis, vascular dementia and Alzheimer's disease, bisphosphonates should play a future role in the prevention and treatment of dementia and Alzheimer's disease.

Hypertension and Interleukin 6

IL-6 is elevated in plasma of preeclamptic women, and twofold elevation of plasma IL-6 increases vascular resistance and arterial pressure in pregnant rats, suggesting a role of the cytokine in hypertension of pregnancy. In one study, the authors tested the hypothesis that IL-6 directly impairs endothelium-dependent relaxation and enhances vascular contraction in systemic vessels of pregnant rats¹⁶⁵. Active stress was measured in aortic strips isolated from virgin and late pregnant Sprague-Dawley rats and then nontreated or treated for 1 h with IL-6 (10 pg/ml to 10 ng/ml). In endothelium-intact vascular strips, phenylephrine (Phe, 10⁻⁵ M) caused an increase in active stress that was smaller in pregnant (4.2±0.3) than virgin rats (5.1±0.3×10⁴ N/m²). IL-6 (1,000 pg/ml) caused enhancement of Phe contraction that was greater in pregnant (10.6±0.7) than virgin rats (7.5±0.4×10⁴ N/m²). The authors concluded that IL-6 inhibits endothelium-dependent NO-cGMP-mediated relaxation and enhances contraction in systemic vessels of virgin and pregnant rats. The greater IL-6-induced inhibition of vascular relaxation and enhancement of contraction in systemic vessels of pregnant rats supports a direct role for IL-6 as one possible mediator of the increased vascular resistance associated with hypertension of pregnancy.

Statins and Hypertension

Recent studies have shown that short-term use of statins can reduce blood pressure (BP) significantly. To determine the long-term effects of statins on BP and aortic stiffness, a single-blind randomized prospective study was performed on 85 hyperlipidemic hypertensive patients whose BP was insufficiently controlled by antihypertensive therapy. Every 3 months, aortic stiffness was assessed by measuring pulse wave velocity (PWV). Patients were randomly allocated to groups treated with pravastatin, simvastatin, fluvastatin, or a nonstatin antihyperlipidemic drug. No significant differences in patient characteristics, kinds of antihypertensive drugs, BP, ankle brachial index, PWV, or serum lipid, creatinine, or C-reactive protein levels were found between the four groups at the start of the study. During the 12-month treatment period, PWV did not change in the pravastatin group or nonstatin group, but it was transiently reduced in the simvastatin group and significantly decreased in the fluvastatin group, even though the doses of the statins used in this study were lower than the usually prescribed dose. All four antihyperlipidemic drugs significantly decreased serum cholesterol levels without affecting BP, ankle brachial index, or serum triglyceride levels. The C-reactive protein serum levels decreased significantly in the three statin groups but not in the nonstatin group. The authors concluded that these results suggest that long-term use of fluvastatin by hyperlipidemic hypertensive patients is associated with a significant reduction in aortic stiffness without any effect on BP¹⁶⁶. Other studies have suggested that lipid-lowering strategies, and particularly statins, could influence blood pressure (BP) control. The aim of the one study was to evaluate the effect of different lipid-lowering strategies on BP control of subjects with hypercholesterolemia who were enrolled in the prospective, population-based, longitudinal Brisighella Heart Study. A total of 1356 subjects with total cholesterol levels >or=239 mg/dL were randomly treated for 5 years (1988-1993) with 1 of these lipid-lowering regimens: low-fat diet, cholestyramine, gemfibrozil, or simvastatin. Participants were divided at baseline into 4 quartiles according to systolic BP level and examined for the percent change in systolic and diastolic BP during the 5 years of treatment. In the study results, a significant decrease in BP was observed in the 2 upper quartiles of systolic BP (>or=140 mm Hg) and was greater in subjects treated with cholesterol-lowering drugs who also had a greater reduction in plasma levels of low-density lipoprotein cholesterol. The BP decrease was greater in patients treated with statin drugs and, among those treated with anti-hypertensive drugs, in subjects in the fourth quartile. The authors concluded that the use of lipid-lowering measures could significantly improve BP control in subjects with both hypercholesterolemia and hypertension. The authors further stated that reduction in BP seems to be enhanced in subjects treated with statins¹⁶⁷.

Bisphosphonates and Hypertension

There is very little literature on the use of bisphosphonates in patients with hypertension. Considering the role of Cholesterol in atherosclerosis, bisphosphonates should play a future role in the prevention and treatment of hypertension.

Age-Related Disorders and Interleukin 6

Overproduction of IL-6, a pro-inflammatory cytokine, is associated with a spectrum of age-related conditions including cardiovascular disease, osteoporosis, arthritis, type 2 diabetes, certain cancers, periodontal disease, frailty, and functional decline. To describe the pattern of change in IL-6 over 6 years among older adults undergoing a chronic stressor, this longitudinal community study assessed the relationship between chronic stress and IL-6 production in 119 men and women who were caregiving for a spouse with dementia and 106 noncaregivers, with a mean age at study entry of 70.58 (SD=8.03) for the full sample. On entry into this portion of the longitudinal study, 28 of the caregivers' spouses had already died, and an additional 50 of the 119 spouses died during the 6 years of this study. Levels of IL-6 and health behaviors associated with IL-6 were measured across 6 years. Caregivers' average rate of increase in IL-6 was about four times as large as that of noncaregivers. Moreover, the mean annual changes in IL-6 among former caregivers did not differ from that of current caregivers even several years after the death of the impaired spouse. There were no systematic group differences in chronic health problems, medications, or health-relevant behaviors that might have accounted for caregivers' steeper IL-6 slope. These data provide evidence of a key mechanism through which chronic stressors may accelerate risk of a host of age-related diseases by prematurely aging the immune response.¹⁶⁸

Age-Related Disorders and Statins

Considering the role of Cholesterol in age-related disorders such as osteoporosis, arthritis, type 2 diabetes, dementia and Alzheimer's disease, statins should play a future role in the prevention and treatment of age-related disorders.

Age-Related Disorders and Bisphosphonates

Considering the role of Cholesterol in age-related disorders such as osteoporosis, arthritis, type 2 diabetes, dementia and Alzheimer's disease, bisphosphonates should play a future role in the prevention and treatment of age-related disorders.

Clinical Implications of Chronic Isoprenoid Suppression and Inhibition of IL-6-Mediated Inflammation

There are currently no large clinical studies utilizing combination of statins and bisphosphonates to synergistically deplete isoprenoids and inhibit Interleukin-6 mediated inflammation. There have been large clinical studies utilizing either statins or bisphosphonates. Some of the patients in these studies may have been on both statins and bisphosphonates. Evidence of safety and efficacy of combination treatment with statins and bisphosphonates may be sought from new clinical trials or sub-group analyses or meta-analyses of existing studies.

The statin studies have shown that statins may decrease the progression of coronary artery disease^{169 170}, reduce the risks of heart attack and death^{171 172 173 174 175 176} lower the risk of stroke in people with coronary artery disease¹⁷⁷. The Prospective Pravastatin Pooling Project (PPP) looked at the long-term safety and efficacy of statins in secondary prevention, based on pooled results from three key statin trials. PPP revealed a highly significant relative risk reduction in total mortality, fatal and nonfatal coronary events, and stroke events in patients with a broad range of patient characteristics¹⁷⁸. The trial demonstrated that pravastatin has a similar incidence of muscle-related side effects as placebo¹⁷⁹. The Collaborative AtoRvastatin Diabetes Study (CARDS) showed patients with type 2 diabetes who received atorvastatin 10 mg daily for four years had a 37% relative risk reduction in the primary endpoint (acute coronary heart disease death, fatal or non-fatal myocardial infarction, unstable angina requiring hospital admission, resuscitated cardiac arrest, coronary revascularisation procedures and stroke)¹⁸⁰. The trial was terminated over a year early on account of a clear benefit demonstrated for the intervention group. Numerous large-scale clinical trials have consistently demonstrated a positive safety and tolerability profile for statins. Non-life-threatening side effects may occur in up to 15% of patients receiving one statin. More serious side effects that may require discontinuation of statin therapy may also occur but at much lower rates. These include significant elevations in the activity of serum aminotransferase and creatine kinase alone or in combination with muscle pain¹⁸¹. The safety of statins in children and adolescents has not yet been well documented.

Bisphosphonates are widely used in osteoporosis and other bone diseases. Large clinical trials have established the strong safety and tolerability profile of bisphosphonates^{182 183}. In the Fracture Intervention Trial (FIT)^{184 185}, administration of alendronate to postmenopausal women with low femoral bone mineral density (BMD) increased spinal BMD to 8 percent over baseline, with a 50 percent decrease in the risk of new vertebral, hip and wrist fractures in women with at least one preexisting vertebral fracture at baseline. The bisphosphonates have minimal non-skeletal toxicity because they bind to bone and are not taken up by other tissues¹⁸⁶. The reduction in renal function that occurs in animal models with administration of high-dosage parenteral bisphosphonate has not occurred in clinical practice. However, because bisphosphonates are excreted through glomerular filtration, intravenous administration of large dosages of pamidronate to patients with severe chronic renal failure or patients on dialysis may be accompanied by marked hypocalcemia and/or hypophosphatemia with associated tetany.³⁹ Iritis, muscle aches and fever can also accompany intravenous bisphosphonate administration and is reversible on discontinuation. Oral bisphosphonates seem to induce serious esophagitis in some patients, may result in gastritis and cause diarrhea.⁴⁰. When used as recommended, serious esophageal complications are few. Patients with known esophageal disease (e.g., achalasia, stricture, Barrett's esophagus, severe reflux and scleroderma) should avoid taking oral bisphosphonates.

### CONCLUSION

In conclusion, we have described the biochemical pathway from cholesterol synthesis to interleukin 6 mediated inflammation. It is our theory that Interleukin 6 mediated inflammation is a common causative factor for atherosclerosis, peripheral vascular disease, coronary artery disease, aging and age-related disorders including osteoporosis, type 2 diabetes, dementia and some forms of arthritis and tumors. We have clarified the relationship between these common illnesses and we determine that pleiotropic effects of bisphosphonates and statins are mediated by isoprenoid depletion and inhibition of Interleukin 6 mediated inflammation.

Isoprenoids, which are intermediates, generated in the cholesterol biosynthesis pathway, play a more significant role than the end product cholesterol, in activation of Interleukin 6 mediated inflammation. Isoprenoids are generated by endogenous cellular cholesterol synthesis in the body as well as by cholesterol synthesis in activated monocytes during the inflammatory response. Isoprenoids are an integral component of the signaling pathway for Interleukin 6 mediated inflammation.

Inhibition of the signal transduction pathway for Interleukin 6 mediated inflammation is key to the prevention and treatment of atherosclerosis, peripheral vascular disease, coronary artery disease, and age-related disorders including osteoporosis, type 2 diabetes, dementia and some forms of arthritis and tumors. Inhibition of Interleukin 6 mediated inflammation may be achieved indirectly through regulation of endogenous cholesterol synthesis and isoprenoid depletion or by direct inhibition of the signal transduction pathway including interleukin-6 inhibitor/antibody, interleukin-6 receptor inhibitor/antibody, gp130 protein inhibitor/antibody, tyrosine kinases inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof. Prompt treatment of infection such as in periodontal disease will prevent or reduce the generation of isoprenoids and induction of Interleukin-6 mediated inflammation by activated monocytes.

Statins and Bisphosphonates have similar mechanisms of action and act on similar diseases in the following ways:

1. Statins and Bisphosphonates inhibit the Mevalonate to Cholesterol conversion pathway and cause isoprenoid depletion; Statins inhibit the enzyme HMG-CoA reductase and Bisphosphonates inhibit the enzyme FPP Synthase.

2. Statins and Bisphosphonates deplete isoprenoids and inhibit the JAK/STAT3 signaling pathway for Interleukin 6 mediated inflammation.

3. Statins and Bisphosphonates have common pleiotropic effects and decrease the progression of atherosclerotic vascular disease and inhibit bone resorption.

4. Combination treatment with agents that inhibit different aspects of the signal transduction pathways for interleukin 6 mediated inflammation, including statins and bisphosphonates, may have better efficacy with fewer side effects in the prevention and treatment of atherosclerosis, peripheral vascular disease, coronary artery disease, and age-related disorders including osteoporosis, type 2 diabetes, dementia and some forms of arthritis and tumors. Evidence of safety and efficacy of combination treatment with inhibitors of Interleukin-6 mediated inflammation should be sought from new clinical trials or sub-group analyses or meta-analyses of existing studies.

Statins, Bisphosphonates are just indirect inhibitors of Interleukin-6 inflammation but yet both class of drugs have enabled a significant decrease in mortality and morbidity from these common illnesses. Newer therapies and drugs will be interleukin-6 inhibitor/antibody, interleukin-6 receptor inhibitor/antibody, gp130 protein inhibitor/antibody, tyrosine kinases inhibitors/antibodies, mitogen-activated protein (MAP) kinase inhibitors/antibodies STAT transcription factors inhibitors/antibodies, altered IL-6, partial peptides of IL-6 or IL-6 receptor, or SOCS (suppressors of cytokine signaling) protein, or a functional fragment thereof.

The public health significance of such new drugs will be difficult to quantify.

It will be apparent to those skilled in the art that variations and modifications to the specific embodiments disclosed herein may be made without departing from the scope of the invention.

I claim:

1. A method of prevention and treatment of vascular and age-related disorders by synergistic inhibition or reduction of Interleukin-6 inflammation in a human or other animal subject. Inhibition of Interleukin 6 mediated inflammation may be achieved indirectly through regulation of endogenous cholesterol synthesis and isoprenoid depletion or by direct inhibition of the signal transduction pathway including interleukin-6 inhibitor/antibody, interleukin-6 receptor inhibitor/antibody, gp130 protein inhibitor/antibody, tyrosine kinases inhibitors/antibodies, mitogen-activated protein (MAP) kinase inhibitors/antibodies, STAT transcription factors inhibitors/antibodies, altered IL-6, partial peptides of IL-6 or IL-6 receptor, or SOCS (suppressors of cytokine signaling) protein, or a functional fragment thereof. Said method comprises administering, to said subject, any one of the following combinations of components that are inhibitors of interleukin-6 mediated inflammation:
I. A and B
II. A, B, and C
III. A and C
IV. B and C
Wherein
A is an inhibitor of cholesterol synthesis and includes one or several Statins and Bisphosphonates selected from the Statin group including of lovastatin, simvastatin, pravastatin, fluvastatin, atorvastatin, rivastatin, red yeast rice, red yeast grain, red yeast powder and other statins or a pharmaceutically acceptable salt thereof and the Bisphosphonate group including of Pamidronate, Etidronate, Clodronate, Alendronate, phosphonic acid derivatives, an ester thereof, a pharmaceutically acceptable salt thereof, a hydrate thereof
B is one or several inhibitors or antibodies of the Interleukin-6 (IL-6) signal transduction pathway including interleukin-6 inhibitor or antibody, interleukin-6 receptor inhibitor or antibody, gp130 protein inhibitor or antibody, Janus kinases inhibitors or antibodies, MAP kinase inhibitors/antibodies, STAT transcription factors inhibitors or antibodies, altered IL-6, partial peptides of IL-6 or IL-6 receptor, or SOCS (suppressors of cytokine signaling) protein, or a functional fragment thereof.
C. is a cholesterol lowering agent or technique selected from the group including of (i) low cholesterol or low fat diet (ii) sequestrants (cholestyramine, colestipol and dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and benzafibrate), (v) inhibitors of cholesterol absorption for example beta-sitosterol and (acyl CoA:cholesterol acyltransferase) inhibitors for example melinamide and (vi) probucol, an ester thereof, a pharmaceutically acceptable salt thereof, a hydrate thereof.
said components being administered simultaneously or separately, in amounts which have the effect of ameliorating the vascular and age-related disorders.

2. The method of claim 1, wherein;
a) said vascular and age-related disorder is atherosclerosis.
b) a therapeutically effective amount of said component or combination of inhibitors of interleukin-6 mediated inflammation is administered subcutaneously, intramuscularly, intravenously, orally or rectally

3. The method of claim 1, wherein;
a) said vascular and age-related disorder is peripheral vascular disease.
b) a therapeutically effective amount of said component or combination of inhibitors of interleukin-6 mediated inflammation is administered subcutaneously, intramuscularly, intravenously, orally or rectally

4. The method of claim 1, wherein;
a) said vascular and age-related disorder is coronary artery disease.
b) a therapeutically effective amount of said component or combination of inhibitors of interleukin-6 mediated inflammation is administered subcutaneously, intramuscularly, intravenously, orally or rectally

5. The method of claim 1, wherein;
a) said vascular and age-related disorder is osteoporosis.
b) a therapeutically effective amount of said component or combination of inhibitors of interleukin-6 mediated inflammation is administered subcutaneously, intramuscularly, intravenously, orally or rectally.

6. The method of claim 1, wherein;
a) said vascular and age-related disorder is arthritis.
b) a therapeutically effective amount of said component or combination of inhibitors of interleukin-6 mediated inflammation is administered subcutaneously, intramuscularly, intravenously, orally or rectally.

7. The method of claim 1, wherein;
a) said vascular and age-related disorder is Type 1 diabetes, Type 2 diabetes, inadequate glucose tolerance or insulin resistance.
b) a therapeutically effective amount of said component or combination of inhibitors of interleukin-6 mediated inflammation is administered subcutaneously, intramuscularly, intravenously, orally or rectally.

8. The method of claim 1, wherein;
a) said vascular and age-related disorder is obesity
b) a therapeutically effective amount of said component or combination of inhibitors of interleukin-6 mediated inflammation is administered subcutaneously, intramuscularly, intravenously, orally or rectally.

9. The method of claim 1, wherein;
a) said vascular and age-related disorder is hypertension
b) a therapeutically effective amount of said component or combination of inhibitors of interleukin-6 mediated inflammation is administered subcutaneously, intramuscularly, intravenously, orally or rectally.

10. The method of claim 1, wherein;
a) said vascular and age-related disorder is dementia
b) a therapeutically effective amount of said component or combination of inhibitors of interleukin-6 mediated inflammation is administered subcutaneously, intramuscularly, intravenously, orally or rectally.

11. The method of claim 1, wherein;
a) said vascular and age-related disorder is Alzheimer's disease
b) a therapeutically effective amount of said component or combination of inhibitors of interleukin-6 mediated inflammation is administered subcutaneously, intramuscularly, intravenously, orally or rectally.

12. The method of claim 1, wherein;
a) said vascular and age-related disorder is Aging
b) a therapeutically effective amount of said component or combination of inhibitors of interleukin-6 mediated inflammation is administered subcutaneously, intramuscularly, intravenously, orally or rectally.

13. The method of claim 1, wherein;
a) said vascular and age-related disorder is Periodontal disease or other chronic low grade infection such as Chlamydia pneumoniae
b) a therapeutically effective amount of said component or combination of inhibitors of interleukin-6 mediated inflammation is administered subcutaneously, intramuscularly, intravenously, orally or rectally.

14. The method of claim 1, wherein;
a) said vascular and age-related disorder is cancer or tumors including multiple myeloma.
b) a therapeutically effective amount of said component or combination of inhibitors of interleukin-6 mediated inflammation is administered subcutaneously, intramuscularly, intravenously, orally or rectally.

15. A method of prevention and treatment of vascular and age-related disorders by inhibition or reduction of Interleukin-6 inflammation through regulation of cholesterol metabolism and isoprenoid depletion, in a human or other animal subject. Said method comprises administering, to said subject, in amounts which have the effect of ameliorating the vascular and age-related disorders, a HMG-CoA reductase inhibitor selected from the group including of lovastatin, simvastatin, pravastatin, fluvastatin, atorvastatin, rivastatin, red yeast rice, red yeast grain, red yeast powder and other statins or a pharmaceutically acceptable salt thereof.

16. The method of claim 15, wherein;
a) said vascular and age-related disorder is peripheral vascular disease.
b) a therapeutically effective amount of said regulator of cholesterol metabolism and interleukin-6 mediated inflammation is administered subcutaneously, intramuscularly, intravenously, orally or rectally.

17. The method of claim 15, wherein;
a) said vascular and age-related disorder is coronary artery disease.
b) a therapeutically effective amount of said regulator of cholesterol metabolism and interleukin-6 mediated inflammation is administered subcutaneously, intramuscularly, intravenously, orally or rectally.

18. The method of claim 15, wherein;
a) said vascular and age-related disorder is arthritis.
b) a therapeutically effective amount of said regulator of cholesterol metabolism and interleukin-6 mediated inflammation is administered subcutaneously, intramuscularly, intravenously, orally or rectally.

19. The method of claim 15, wherein;
a) said vascular and age-related disorder is Type 1 diabetes, Type 2 diabetes, inadequate glucose tolerance or insulin resistance.
b) a therapeutically effective amount of said regulator of cholesterol metabolism and interleukin-6 mediated inflammation is administered subcutaneously, intramuscularly, intravenously, orally or rectally.

20. The method of claim 15, wherein;
a) said vascular and age-related disorder is obesity
b) a therapeutically effective amount of said regulator of cholesterol metabolism and interleukin-6 mediated inflammation is administered subcutaneously, intramuscularly, intravenously, orally or rectally.

21. The method of claim 15, wherein;
a) said vascular and age-related disorder is hypertension
b) a therapeutically effective amount of said regulator of cholesterol metabolism and interleukin-6 mediated inflammation is administered subcutaneously, intramuscularly, intravenously, orally or rectally.

22. The method of claim 15, wherein;
a) said vascular and age-related disorder is dementia
b) a therapeutically effective amount of said regulator of cholesterol metabolism and interleukin-6 mediated inflammation is administered subcutaneously, intramuscularly, intravenously, orally or rectally.

23. The method of claim 15, wherein;
a) said vascular and age-related disorder is Alzheimer's disease
b) a therapeutically effective amount of said regulator of cholesterol metabolism and interleukin-6 mediated inflammation is administered subcutaneously, intramuscularly, intravenously, orally or rectally.

24. The method of claim 15, wherein;
a) said vascular and age-related disorder is Aging
b) a therapeutically effective amount of said regulator of cholesterol metabolism and interleukin-6 mediated inflammation is administered subcutaneously, intramuscularly, intravenously, orally or rectally.

25. The method of claim 15, wherein;
a) said vascular and age-related disorder is Periodontal disease or other chronic low grade infection such as Chlamydia pneumoniae
b) a therapeutically effective amount of said component or combination of inhibitors of interleukin-6 mediated inflammation is administered subcutaneously, intramuscularly, intravenously, orally or rectally.

26. The method of claim 15, wherein;
a) said vascular and age-related disorder is cancer or tumors including multiple myeloma.
b) a therapeutically effective amount of said component or combination of inhibitors of interleukin-6 mediated inflammation is administered subcutaneously, intramuscularly, intravenously, orally or rectally.

27. A method of prevention and treatment of vascular and age-related disorders by inhibition or reduction of Interleukin-6 inflammation through regulation of cholesterol metabolism and isoprenoid depletion, in a human or other animal subject. Said method comprises administering, to said subject, in amounts which have the effect of ameliorating the vascular and age-related disorders, a bisphosphonate selected from the group including of Pamidronate, Etidronate, Clodronate, Alendronate, phosphonic acid derivatives, an ester thereof, a pharmaceutically acceptable salt thereof, a hydrate thereof.

28. The method of claim 27, wherein;
a) said vascular and age-related disorder is peripheral vascular disease.
b) a therapeutically effective amount of said regulator of cholesterol metabolism and interleukin-6 mediated inflammation is administered subcutaneously, intramuscularly, intravenously, orally or rectally.

29. The method of claim 27, wherein;
a) said vascular and age-related disorder is coronary artery disease.
b) a therapeutically effective amount of said regulator of cholesterol metabolism and interleukin-6 mediated inflammation is administered subcutaneously, intramuscularly, intravenously, orally or rectally.

30. The method of claim 27, wherein;
a) said vascular and age-related disorder is arthritis.
b) a therapeutically effective amount of said regulator of cholesterol metabolism and interleukin-6 mediated inflammation is administered subcutaneously, intramuscularly, intravenously, orally or rectally.

31. The method of claim 27, wherein;
a) said vascular and age-related disorder is Type 1 diabetes, Type 2 diabetes, inadequate glucose tolerance or insulin resistance.
b) a therapeutically effective amount of said regulator of cholesterol metabolism and interleukin-6 mediated inflammation is administered subcutaneously, intramuscularly, intravenously, orally or rectally.

32. The method of claim 27, wherein;
a) said vascular and age-related disorder is obesity
b) a therapeutically effective amount of said regulator of cholesterol metabolism and interleukin-6 mediated inflammation is administered subcutaneously, intramuscularly, intravenously, orally or rectally.

33. The method of claim 27, wherein;
a) said vascular and age-related disorder is hypertension
b) a therapeutically effective amount of said regulator of cholesterol metabolism and interleukin-6 mediated inflammation is administered subcutaneously, intramuscularly, intravenously, orally or rectally.

34. The method of claim 27, wherein;
a) said vascular and age-related disorder is dementia
b) a therapeutically effective amount of said regulator of cholesterol metabolism and interleukin-6 mediated inflammation is administered subcutaneously, intramuscularly, intravenously, orally or rectally.

35. The method of claim 27, wherein;
a) said vascular and age-related disorder is Alzheimer's disease
b) a therapeutically effective amount of said regulator of cholesterol metabolism and interleukin-6 mediated inflammation is administered subcutaneously, intramuscularly, intravenously, orally or rectally.

36. The method of claim 27, wherein;
a) said vascular and age-related disorder is Aging
b) a therapeutically effective amount of said regulator of cholesterol metabolism and interleukin-6 mediated inflammation is administered subcutaneously, intramuscularly, intravenously, orally or rectally.

37. The method of claim 27, wherein;
a) said vascular and age-related disorder is Periodontal disease or other chronic low grade infection such as Chlamydia pneumoniae
b) a therapeutically effective amount of said component or combination of inhibitors of interleukin-6 mediated inflammation is administered subcutaneously, intramuscularly, intravenously, orally or rectally.

38. The method of claim 27, wherein;
a) said vascular and age-related disorder is cancer or tumors including multiple myeloma.
b) a therapeutically effective amount of said component or combination of inhibitors of interleukin-6 mediated inflammation is administered subcutaneously, intramuscularly, intravenously, orally or rectally.

39. A method of prevention and treatment of vascular and age-related disorders by inhibition or reduction of Interleukin-6 inflammation through regulation of cholesterol metabolism and isoprenoid depletion, in a human or other animal subject. Said method comprises administering, to said subject, in amounts which have the effect of ameliorating the vascular and age-related disorders, a cholesterol lowering agent or technique selected from the group including of (i) low cholesterol or low fat diet (ii) sequestrants (cholestyramine, colestipol and dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and benzafibrate), (v) inhibitors of cholesterol absorption for example beta-sitosterol and (acyl CoA:cholesterol acyltransferase) inhibitors for example melinamide and (vi) probucol, an ester thereof, a pharmaceutically acceptable salt thereof, a hydrate thereof.

40. The method of claim 39, wherein;
a) said vascular and age-related disorder is peripheral vascular disease.
b) a therapeutically effective amount of said regulator of cholesterol metabolism and interleukin-6 mediated inflammation is administered subcutaneously, intramuscularly, intravenously, orally or rectally.

41. The method of claim 39, wherein;
a) said vascular and age-related disorder is coronary artery disease.
b) a therapeutically effective amount of said regulator of cholesterol metabolism and interleukin-6 mediated inflammation is administered subcutaneously, intramuscularly, intravenously, orally or rectally.

42. The method of claim 39, wherein;
a) said vascular and age-related disorder is arthritis.
b) a therapeutically effective amount of said regulator of cholesterol metabolism and interleukin-6 mediated inflammation is administered subcutaneously, intramuscularly, intravenously, orally or rectally.

43. The method of claim 39, wherein;
a) said vascular and age-related disorder is Type 1 diabetes, Type 2 diabetes, inadequate glucose tolerance or insulin resistance.
b) a therapeutically effective amount of said regulator of cholesterol metabolism and interleukin-6 mediated inflammation is administered subcutaneously, intramuscularly, intravenously, orally or rectally.

44. The method of claim 39, wherein;
a) said vascular and age-related disorder is obesity
b) a therapeutically effective amount of said regulator of cholesterol metabolism and interleukin-6 mediated inflammation is administered subcutaneously, intramuscularly, intravenously, orally or rectally.

45. The method of claim 39, wherein;
a) said vascular and age-related disorder is hypertension
b) a therapeutically effective amount of said regulator of cholesterol metabolism and interleukin-6 mediated inflammation is administered subcutaneously, intramuscularly, intravenously, orally or rectally.

46. The method of claim 39, wherein;
a) said vascular and age-related disorder is dementia
b) a therapeutically effective amount of said regulator of cholesterol metabolism and interleukin-6 mediated inflammation is administered subcutaneously, intramuscularly, intravenously, orally or rectally.

47. The method of claim 39, wherein;
a) said vascular and age-related disorder is Alzheimer's disease
b) a therapeutically effective amount of said regulator of cholesterol metabolism and interleukin-6 mediated inflammation is administered subcutaneously, intramuscularly, intravenously, orally or rectally.

48. The method of claim 39, wherein;
a) said vascular and age-related disorder is Aging
b) a therapeutically effective amount of said regulator of cholesterol metabolism and interleukin-6 mediated inflammation is administered subcutaneously, intramuscularly, intravenously, orally or rectally.

49. The method of claim 39, wherein;
a) said vascular and age-related disorder is Periodontal disease or other chronic low grade infection such as Chlamydia pneumoniae
b) a therapeutically effective amount of said component or combination of inhibitors of interleukin-6 mediated inflammation is administered subcutaneously, intramuscularly, intravenously, orally or rectally.

50. The method of claim 39, wherein;
a) said vascular and age-related disorder is cancer or tumors including multiple myeloma.
b) a therapeutically effective amount of said component or combination of inhibitors of interleukin-6 mediated inflammation is administered subcutaneously, intramuscularly, intravenously, orally or rectally.

51. A method of prevention and treatment of vascular and age-related disorders by inhibition or reduction of Interleukin-6 inflammation in a human or other animal subject. Said method comprises administering, to said subject, in amounts which have the effect of ameliorating the vascular and age-related disorders, one or several inhibitors or antibodies of the Interleukin-6 (IL-6) signal transduction pathway including interleukin-6 inhibitor or antibody, interleukin-6 receptor inhibitor or antibody, gp130 protein inhibitor or antibody, Janus kinases inhibitors or antibodies, MAP kinase inhibitors/antibodies, STAT transcription factors inhibitors or antibodies, altered IL-6, partial peptides of IL-6 or IL-6 receptor, or SOCS (suppressors of cytokine signaling) protein, or a functional fragment thereof.

52. The method of claim 51, wherein;
a) said vascular and age-related disorder is peripheral vascular disease.
b) a therapeutically effective amount of said component or combination of inhibitors of interleukin-6 mediated inflammation is administered subcutaneously, intramuscularly, intravenously, orally or rectally.

53. The method of claim 51, wherein;
a) said vascular and age-related disorder is coronary artery disease.
b) a therapeutically effective amount of said component or combination of inhibitors of interleukin-6 mediated inflammation is administered subcutaneously, intramuscularly, intravenously, orally or rectally.

54. The method of claim 51, wherein;
a) said vascular and age-related disorder is arthritis.
b) a therapeutically effective amount of said component or combination of inhibitors of interleukin-6 mediated inflammation is administered subcutaneously, intramuscularly, intravenously, orally or rectally.

55. The method of claim 51, wherein;
a) said vascular and age-related disorder is Type 1 diabetes, Type 2 diabetes, inadequate glucose tolerance or insulin resistance.
b) a therapeutically effective amount of said component or combination of inhibitors of interleukin-6 mediated inflammation is administered subcutaneously, intramuscularly, intravenously, orally or rectally.

56. The method of claim 51, wherein;
a) said vascular and age-related disorder is obesity
b) a therapeutically effective amount of said component or combination of inhibitors of interleukin-6 mediated inflammation is administered subcutaneously, intramuscularly, intravenously, orally or rectally.

57. The method of claim 51, wherein;
a) said vascular and age-related disorder is hypertension
b) a therapeutically effective amount of said component or combination of inhibitors of interleukin-6 mediated inflammation is administered subcutaneously, intramuscularly, intravenously, orally or rectally.

58. The method of claim 51, wherein;
a) said vascular and age-related disorder is dementia
b) a therapeutically effective amount of said component or combination of inhibitors of interleukin-6 mediated inflammation is administered subcutaneously, intramuscularly, intravenously, orally or rectally.

59. The method of claim 51, wherein;
a) said vascular and age-related disorder is Alzheimer's disease
b) a therapeutically effective amount of said component or combination of inhibitors of interleukin-6 mediated inflammation is administered subcutaneously, intramuscularly, intravenously, orally or rectally.

60. The method of claim 51, wherein;
a) said vascular and age-related disorder is Aging
b) a therapeutically effective amount of said component or combination of inhibitors of interleukin-6 mediated inflammation is administered subcutaneously, intramuscularly, intravenously, orally or rectally.

61. The method of claim 51, wherein;
a) said vascular and age-related disorder is Periodontal disease or other chronic low grade infection such as Chlamydia pneumoniae
b) a therapeutically effective amount of said component or combination of inhibitors of interleukin-6 mediated inflammation is administered subcutaneously, intramuscularly, intravenously, orally or rectally.

62. The method of claim 51, wherein;
a) said vascular and age-related disorder is cancer or tumors including multiple myeloma.
b) a therapeutically effective amount of said component or combination of inhibitors of interleukin-6 mediated inflammation is administered subcutaneously, intramuscularly, intravenously, orally or rectally.
